(19) United States
(12) Patent Application Publication
(10) Pub. No.: US 2007/0031328 A1
(43) Pub. Date: Feb. 8, 2007

(54) Radiolabeled-pegylation of ligands for use as imaging agents

(76) Inventor: Hank F. Kung, Wynnewood, PA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/474,489

(22) Filed: Jun. 26, 2006

Related U.S. Application Data
(60) Provisional application No. 60/693,501, filed on Jun. 24, 2005.

(51) Int. Cl. A61K 51/00

(52) U.S. Cl. 424/1.11

(57) ABSTRACT

The present invention is directed to a method of using radiolabeled ethylene glycol (n=1) (EG) or polyethylene glycol (n=from 2 to 10) (PEG) as a labeling group moiety on compounds that can be useful for imaging tissues. Specifically, the EG or PEG moiety preferably contains a radiofluorine (¹⁸F), and is covalently bonded to a ligand (L). The L portion of the molecule can be any molecule appropriate for covalently bonding with the radiolabeled EG or PEG moiety and subsequent use as an imaging agent. In particular, the imaging agent is preferably an agent suitable for administering to a mammal and detecting by PET or SPECT imaging.

### BACKGROUND OF THE INVENTION

1. Field of the Invention

This invention relates to bioactive compounds, methods of diagnostic imaging using radiolabeled compounds, and methods of making radiolabeled compounds.

2. Background Art

A number of approaches have been developed for noninvasive measurements of tissue in vivo. These approaches have generally used techniques of nuclear medicine to generate images of a variety of tissues, organs, receptors, etc. These imaging methods include positron emission tomography (PET) and single photon emission computed tomography (SPECT).

Single photon emission computerized tomography (SPECT) and positron emission tomography (PET) are well known nuclear imaging systems in medicine. Generally, in nuclear imaging, a radioactive isotope is injected into, inhaled by or ingested by a patient. The isotope, provided as a radioactive-labeled pharmaceutical (radio-pharmaceutical) is chosen based on bio-kinetic properties that cause preferential uptake by different tissues. The gamma photons emitted by the radio-pharmaceutical are detected by radiation detectors outside the body, giving its spatial and uptake distribution within the body, with little trauma to the patient.

SPECT and PET imaging couple conventional planar nuclear imaging techniques and tomographic reconstruction methods. Gamma cameras, arranged in a specific geometric configuration, are mounted on a gantry that rotates them around a patient, to acquire data from different angular views. Projection (or planar) data acquired from different views are reconstructed, using image reconstruction methods, to generate cross-sectional images of the internally distributed radio-pharmaceuticals. These images provide enhanced contrast and greater detail, when compared with planer images obtained with conventional nuclear imaging methods.

Noninvasive, nuclear imaging techniques can be used to obtain basic and diagnostic information about the physiology and biochemistry of a variety of living subjects including experimental animals, normal humans and patients. These techniques rely on the use of sophisticated imaging instrumentation which is capable of detecting radiation emitted from radiotracers administered to such living subjects. The information obtained can be reconstructed to provide planar and tomographic images which reveal distribution of the radiotracer as a function of time. Use of appropriately designed radiotracers can result in images which contain information on the structure, function and most importantly, the physiology and biochemistry of the subject. Much of this information cannot be obtained by other means. The radiotracers used in these studies are designed to have defined behaviors in vivo which permit the determination of specific information concerning the physiology or biochemistry of the subject or the effects that various diseases or drugs have on the physiology or biochemistry of the subject. Currently, radio-tracers are available for obtaining useful information concerning such things as cardiac function, myocardial blood flow, lung perfusion, liver function, brain blood flow, regional brain glucose and oxygen metabolism.

Compounds can be labeled with either positron or gamma emitting radionuclides. For imaging, the most commonly used positron emitting radionuclides are ¹¹C, ¹⁸F, ¹⁵O and ¹³N, which have half lives of 20, 110, 2 and 10 min. respectively. Several gamma emitting radiotracers are available. The most widely used of these include ^{99m} Tc and ¹²³I.

Amyloidosis is a condition characterized by the accumulation of various insoluble, fibrillar proteins in the tissues of a patient. An amyloid deposit is formed by the aggregation of amyloid proteins, followed by the further combination of aggregates and/or amyloid proteins.

In addition to the role of amyloid deposits in Alzheimer's disease, the presence of amyloid deposits has been shown in diseases such as Mediterranean fever, Muckle-Wells syndrome, idiopathic myeloma, amyloid polyneuropathy, amyloid cardiomyopathy, systemic senile amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, Isolated atrial amyloid, β2-microglobulin amyloid in dialysis patients, inclusion body myositis, β2-amyloid deposits in muscle wasting disease, and Islets of Langerhans diabetes Type II insulinoma.

Thus, a simple, noninvasive method for detecting and quantitating amyloid deposits in a patient has been eagerly sought. Presently, detection of amyloid deposits involves histological analysis of biopsy or autopsy materials. Both methods have drawbacks. For example, an autopsy can only be used for a postmortem diagnosis.

The direct imaging of amyloid deposits in vivo is difficult, as the deposits have many of the same physical properties (e.g., density and water content) as normal tissues. Attempts to image amyloid deposits using magnetic resonance imaging (MRI) and computer-assisted tomography (CAT) have been disappointing and have detected amyloid deposits only under certain favorable conditions. In addition, efforts to label amyloid deposits with antibodies, serum amyloid P protein, or other probe molecules have provided some selectivity on the periphery of tissues, but have provided for poor imaging of tissue interiors.

Potential ligands for detecting Aβ aggregates in the living brain must cross the intact blood-brain barrier. Thus brain uptake can be improved by using ligands with relatively smaller molecular size (compared to Congo Red) and increased lipophilicity. Highly conjugated thioflavins (S and T) are commonly used as dyes for staining the Aβ aggregates in the AD brain (Elhaddaoui, A., et al., Biospectroscopy 1: 351-356 (1995)). These compounds are based on benzothiazole, which is relatively small in molecular size. However, thioflavins contain an ionic quarternary amine, which is permanently charged and unfavorable for brain uptake.

Thus, it would be useful to have a method of labeling the ligands that also imparts an improved brain bioavailability of the radiolabeled ligands. These ligands would in turn be useful for imaging amyloid in the brain.

### BRIEF SUMMARY OF THE INVENTION

The present invention is directed to a method of using ethylene glycol (n=1) (EG) or polyethylene glycol (n=from 2 to 10) (PEG) as a moiety on compounds that can be useful for imaging tissues. Specifically, the EG or PEG moiety preferably contains a radiofluorine (¹⁸F), radioiodine, or radiometal, and is covalently bonded to a ligand (L). The L portion of the molecule can be any molecule that, 1) binds amyloid deposits, and 2) is appropriate for covalently bonding with the above EG or PEG moiety and subsequent use as an imaging agent. In particular, the imaging agent is preferably an agent suitable for administering to a mammal and detecting by PET or SPECT imaging.

The present invention also provides diagnostic compositions comprising a radiolabeled compound of Formula IV and a pharmaceutically acceptable carrier or diluent.

The invention further provides a method of imaging amyloid deposits in a mammal. The method comprises introducing into a mammal a detectable quantity of a labeled compound of Formula IV or a pharmaceutically acceptable salt, ester, amide, or prodrug thereof.

A further aspect of this invention is directed to methods and intermediates useful for synthesizing the compounds of Formula IV.

### BRIEF DESCRIPTION OF THE FIGURES

FIG. 1 depicts representative compounds of Formula IV, where L is L9 (SB), L1 (IMPY) or L2 (BF and PIB).

FIG. 2 depicts an in vitro autoradiography of brain (cortical section) from a confirmed AD patient labeled with [¹⁸F]5a-c (compounds of Formula IV, where L is L2), showing the distinctive labeling of Aβ (amyloid) plaques with the identified ¹⁸F tracers of the present invention.

FIGS. 3, 4 and 5 depict autoradiographs of brain sections labeled with several compounds of the invention.

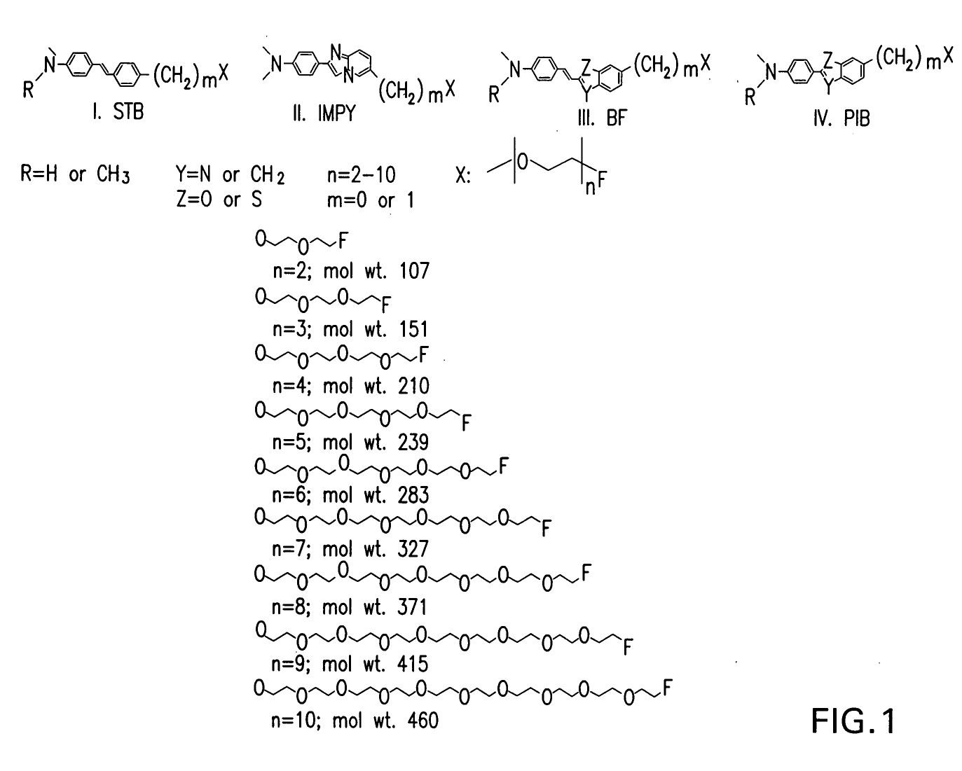

### DETAILED DESCRIPTION OF THE INVENTION

In one aspect, the present invention is directed to a method of labeling compounds with a radiolabeled ethylene glycol (EG) or polyethylene glycol (PEG) chain where the number of ethoxy groups can be from 2 to 10. Preferably, the radiolabeled EG or PEG contains ¹⁸F. The method of labeling can be used to radiolabel any suitable compound that is useful for PET or SPECT imaging.

Useful compounds include any compound for imaging amyloid deposits in the brain. Useful compounds that are also suitable for the present method include compounds that have an appropriate reactive site for combining with a halogenated EG or PEG.

Before adding a radiolabeled or non-radiolabeled EG or PEG moiety of appropriate size as described herein, a suitable compound as described above may already be in use for PET imaging purposes. If the compound is a known imaging agent, the present method would be directed to preparing an alternate imaging agent that contains a EG or PEG chain. An advantage of the present method is that the EG or PEG chain can lower lipophilicity and improve bioavailability. Therefore, in an especially preferred embodiment, the present method is directed to preparing compounds containing a radiolabeled or non-radiolabeled EG or PEG wherein the product of this method has lower lipophilicity and improved bioavailability compared to the starting compound.

Because the EG or PEG moiety can lower lipophilicity and improve bioavailability of the ligand (L), applying this labeling method can yield compounds with improved central nervous system penetration. Thus, this method is particularly useful for labeling compounds that are intended to be used for imaging amyloid deposits in the central nervous system, including specifically the brain. The present method is also particularly useful as a means of improving the bioavailability of brain imaging compounds by increasing their ability to cross the blood-brain-barrier and associate with their intended target.

The present method of preparing the imaging agents comprises,

a) contacting a ligand (L), which contains a first reactive group optionally selected from the group consisting of —OH and —OMs, and all other moieties of similar chemical nature, with a reagent having the following Formula I, 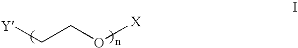
wherein n is an integer from 1 to 10, optionally from 2 to 10; Y′ is a third reactive group, optionally selected from the group consisting of hydrogen or halogen, preferably Br, and X is a second reactive group optionally selected from the group consisting of a halogen, preferably Cl or -trialkylsilane (such as TBS), and all other moieties of similar chemical nature, such that said first reactive group reacts with said second reactive group or the carbon to which it is attached to form a compound of Formula II, 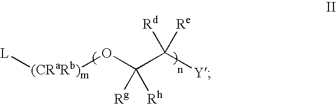
b) contacting a compound of Formula II with a reagent (Z) such as an alkylsulfonate, e.g., MsCl, TsCl, triflate, etc., to prepare a compound of Formula III, 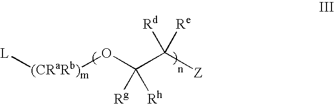
wherein Z is a leaving group, such as —OTs, —OMs or triflate; and c) contacting a compound of Formula III with known radiohalogenating or chelating reagents, preferably TBAF or K222, wherein a radiolabeled ligand having the following Formula IV 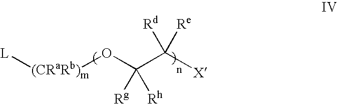
wherein X′ is a radiohalogen or chelating moiety, such as a metal chelating moiety of the N₂S₂ type, is prepared.

One embodiment of the above method comprises, a) contacting a ligand (L-(CR^{a}R^{b})ₘ), wherein R^{a}, R^{b} and m are as described above, said ligand containing a first reactive group, with a compound having the Formula I, wherein n is an integer from 1 to 10, optionally from 2 to 10; Y′ is a third reactive group, and X is a second reactive group such that said first reactive group reacts with said second reactive group or the carbon to which it is attached to form a compound of Formula II, b) contacting a compound of Formula II with a reagent (Z) to prepare a compound of Formula III, wherein Z is a leaving group; and c) contacting a compound of Formula III with a radiohalogenating agent, wherein a radiolabeled ligand of Formula IV as described above is prepared.

The radiohalogenating, chelating reagents and chelating moiety used in the present method are more fully described below.

In each of the Formulae IV, V, VI, VII, VIII, IX, X and XI, described herein the value for X′ can be a halogen, radiohalogen or a chelating moiety capable of complexing with a metal, for example, a N₂S₂ type tetradentate chelating moiety. The following is an example, but is not intended to be limiting of these types of chelating moieties: 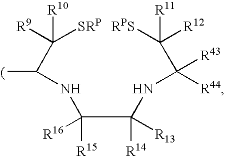
wherein R^{P} is hydrogen, or a sulfhydryl protecting group such as methoxymethyl, methoxyexthoxyethyl, p-methoxybenzyl or benzyl, and R⁹ R¹⁰, R¹¹, R¹², R¹³, R¹⁴, R¹⁵, R¹⁶, R⁴³ and R⁴⁴ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl. When complexed with a metal such as 99m-Tc, -Ch has the following formula:

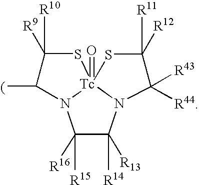

Preferably, the L portion of the imaging agent is a molecule that binds to specific sites or receptors in a mammal that are desirable loci for PET or SPECT imaging such as amyloid deposits. Thus, in a preferred embodiment, the imaging agent comprises a radiolabeled EG or PEG imaging moiety covalently bound to a compound that specifically targets amyloid deposits, such as amyloid aggregates or plaques.

In another aspect, the present invention is directed to the use of the above method for preparing compounds of Formula V, 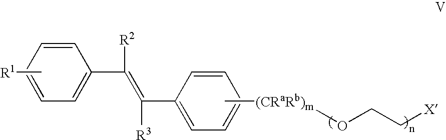
or a pharmaceutically acceptable salt thereof, wherein R¹ is selected from the group consisting of: hydrogen, C₁₋₄ alkyl, amino(C₂₋₄)alkyl, halo(C₁₋₄)alkyl, C₆₋₁₀ aryl, haloarylalkyl, and —NR^{d}R^{e}, wherein R^{d} and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR⁶ in said ring, where R⁶ is hydrogen or C₁₋₄ alkyl; R² and R³, in each instance, is selected from the group consisting of: hydrogen and C₁₋₄ alkyl; R^{a} and R^{b}, in each instance, is selected from the group consisting of: hydrogen, C₁₋₄ alkyl, di- or mono (C₁₋₄)alkylamino, amino(C₂₋₄)alkyl, halo(C₁₋₄)alkyl, hydroxy(C₁₋₁₀)alkyl and haloarylalkyl; m is an integer from 1 to 5; n is an integer from 1 to 10; and X′ is selected from the group consisting of: -Ch, ¹²⁵I, ¹³¹I, ¹²³I, ¹⁸F, ⁷⁶Br, or ⁷⁷Br.

Useful values of m are integers from 1 to 5. Preferably, m is 1 or 2.

Useful values of n are integers from 1 to 10. Preferably, n is an integer from 2 to 5. More preferably, n is 3 or 4.

Useful values of X′ include the chelating moiety and all radiohalogens listed above. More preferably X′ is ¹²³I, ¹²⁵I or ¹⁸F.

Prior to step a) of the present method of preparing a compound of Formula V, the ligand (L) portion contains an appropriate reactive moiety for covalently bonding to the reactant having the structure Formula I. In this aspect, L has the following structure: 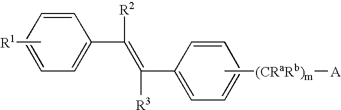
wherein, R^{a}, R^{b}, R¹, R², R³ and m are as described above, and A is an appropriate group for covalently bonding with Formula I.

The ligand portion for preparing a compound of Formula V can be prepared according to methods fully disclosed in published U.S. patent application Ser. No. 10/228,275, herein incorporated by reference in its entirety.

Preferred compounds of Formula V have the following structure: 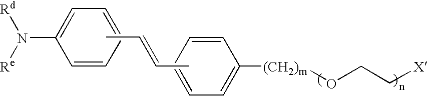
wherein, R^{d} and R^{e}, in each instance, is independently selected from the group consisting of: hydrogen, C₁₋₄ alkyl and halo(C₁₋₄)alkyl; m is an integer from 1 to 5, preferably 1; n is an integer from 2 to 10, preferably 3 or 4; and X′ is selected from the group consisting of: ¹²³I, ¹²⁵I and ¹⁸F.

Compounds of Formula V that are more preferred include those having the structure: 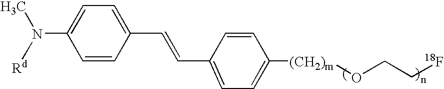
wherein, R^{d} is methyl or hydrogen; m is an integer from 1 to 5, preferably 1; and n is an integer from 2 to 10, preferably 3 or 4.

In another aspect, the present invention is directed to a method of preparing compounds of Formula VI: 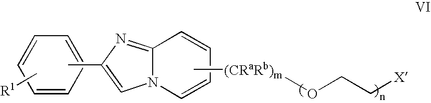
or a pharmaceutically acceptable salt thereof, wherein R¹ is selected from the group consisting of: hydrogen, C₁₋₄ alkyl, amino(C₂₋₄)alkyl, halo(C₁₋₄)alkyl, C₆₋₁₀ aryl, haloarylalkyl, and —NR^{d}R^{e}, wherein R^{d} and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR⁶ in said ring, where R⁶ is hydrogen or C₁₋₄ alkyl; R^{a} and R^{b}, in each instance, is selected from the group consisting of: hydrogen, C₁₋₄ alkyl, di- or mono (C₁₋₄)alkylamino, amino(C₂₋₄)alkyl, halo(C₁₋₄)alkyl, hydroxy(C₁₋₁₀)alkyl and haloarylalkyl; m is an integer from 0 to 4; n is an integer from 1 to 10; and X′ is selected from the group consisting of: -Ch, ¹²⁵I, ¹³¹I, ¹²³I, ¹⁸F, ⁷⁶Br, or ⁷⁷Br.

Useful values of m are integers from 0 to 4. Preferably, m is an integer from 0 to 2. More preferably, m is 0 or 1.

Useful values of n are integers from 1 to 10. Preferably, n is an integer from 2 to 5. More preferably, n is 3 or 4.

Useful values of X′ include the chelating moiety and all radiohalogens listed above. More preferably, X′ is ¹²³I, ¹²¹I or ¹⁸F.

Prior to step a) of the present method of preparing a compound of Formula VI, the ligand (L) portion contains an appropriate reactive moiety for covalently bonding to the reactant having the structure Formula I. In this aspect, L has the following structures: 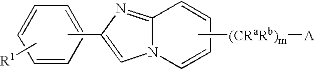
wherein, R^{a}, R^{b}, R¹ and m are as described above, and A is an appropriate group for covalently bonding with Formula I; or preferably, 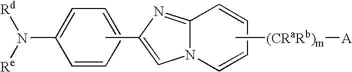
wherein R^{d} and R^{e} are as described above. Examples of appropriate groups include: —OH.

The ligand portion for preparing a compound of Formula VI can be prepared according to methods fully disclosed in U.S. Pat. No. 6,696,039, herein incorporated by reference in its entirety.

Preferred compounds of Formula VI have the following structure: 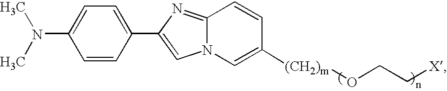
wherein, m is 1 or 2; n is an integer from 2 to 10, preferably 3 or 4; and X′ is preferably ¹²⁵I, ¹²³I or ¹⁸F.

Compounds of Formula VI that are more preferred have the following structure: 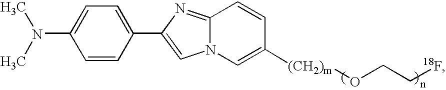
wherein, m is 1 or 2; n is an integer from 2 to 10, preferably 3 or 4.

Another aspect of the present invention is directed to compounds of the following Formula VII: 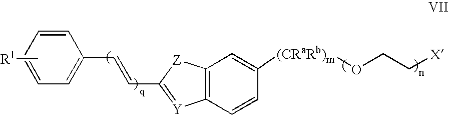
or a pharmaceutically acceptable salt thereof, wherein R¹ is selected from the group consisting of: hydrogen, C₁₋₄ alkyl, amino(C₂₋₄)alkyl, halo(C₁₋₄)alkyl, C₆₋₁₀ aryl, haloarylalkyl, and —NR^{d}R^{e}, wherein R^{d} and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR⁶ in said ring, where R⁶ is hydrogen or C₁₋₄ alkyl; R^{a} and R^{b}, in each instance, is selected from the group consisting of: hydrogen, C₁₋₄ alkyl, di- or mono (C₁₋₄)alkylamino, amino(C₂₋₄)alkyl, halo(C₁₋₄)alkyl, hydroxy(C₁₋₁₀)alkyl and haloarylalkyl; q is an integer from 0 to 3; Z is O, S or N; Y is N or —CH; X′ is selected from the group consisting of: -Ch, ¹²⁵I, ¹³¹I, ¹²³I, ¹⁸F, ⁷⁶Br, or ⁷⁷Br; m is an integer from 0 to 5; and n is an integer from 1 to 10.

Useful values of m are integers from 0 to 5. Preferably, m is an integer from 0 to 2. More preferably, m is 0 or 1.

Useful values of n are integers from 1 to 10. Preferably, n is an integer from 2 to 5. More preferably, n is 3 or 4.

Useful values of X′ include the chelating moiety and all radiohalogens listed above. Preferably, X′ is ¹²³I, ¹²⁵I or ¹⁸F.

Prior to step a) of the present method of preparing a compound of Formula VII, the ligand (L) portion contains an appropriate reactive moiety for covalently bonding to the reactant having the structure Formula I. In this aspect, L has the following structure: 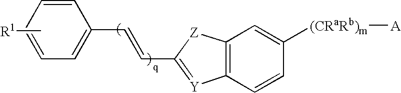
wherein, R^{a}, R^{b}, R¹, m, q, Z and Y are as described above, and A is an appropriate group for covalently bonding with Formula I. Examples of appropriate groups include: —OH.

The ligand portion for preparing a compound of Formula VII can be prepared according to methods fully disclosed in U.S. Pat. Nos. 6,001,331 and 6,696,039 B2.

Preferred compounds of Formula VII have the following structures: 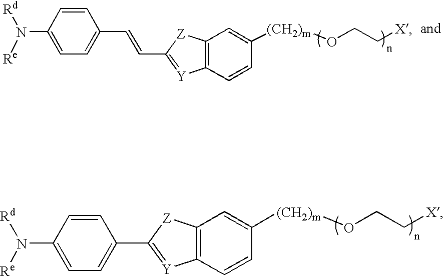
wherein, R^{d} and R^{e}, in each instance, is independently selected from the group consisting of: hydrogen, C₁₋₄ alkyl and halo(C₁₋₄)alkyl; Z is O or S; Y is N or —CH; m is 1 or 2; n is an integer from 2 to 10, preferably 3 or 4; and X′ is ¹²³I, ¹²⁵I or ¹⁸F.

Compounds of Formula VII that are more preferred include: 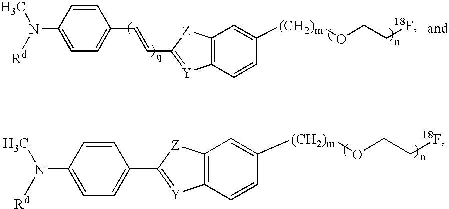
wherein, R^{d} is hydrogen or methyl; Z is O or S; Y is N or —CH; m is 1 or 2; and n is an integer from 2 to 5, preferably 3 or 4, and q, if present, is 1.

In another embodiment, the invention is directed to the preparation of compounds of Formula VIII: 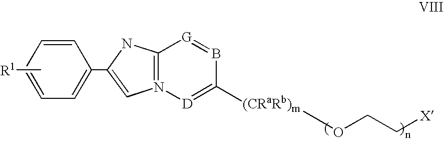
or a pharmaceutically acceptable salt thereof, wherein G, B and D are CH or N, provided that at least one no more than two of G, B and D is N; R¹ is selected from the group consisting of: hydrogen, C₁₋₄ alkyl, amino(C₂₋₄)alkyl, halo(C₁₋₄)alkyl, C₆₋₁₀ aryl, haloarylalkyl, and —NR^{d}R^{e}, wherein R^{d} and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR in said ring, where R⁶ is hydrogen or C₁₋₄ alkyl; R^{a} and R^{b}, in each instance, is selected from the group consisting of: hydrogen, C₁₋₄ alkyl, di- or mono (C₁₋₄)alkylamino, amino(C₂₋₄)alkyl, halo(C₁₋₄)alkyl, hydroxy(C₁₋₁₀)alkyl and haloarylalkyl; X′ is selected from the group consisting of: -Ch, ¹²⁵I, ¹³¹I, ¹²³I, ¹⁸F, ⁷⁶Br, or ⁷⁷Br; m is an integer from 0 to 5; and n is an integer from 1 to 10.

Useful values of m are integers from 0 to 5. Preferably, m is an integer from 0 to 2. More preferably, m is 0 or 1.

Useful values of n are integers from 1 to 10. Preferably, n is an integer from 2 to 5. More preferably, n is 3 or 4.

Useful values of X′ include the chelating moiety and all radiohalogens listed above. Preferably, X′ is ¹²³I, ¹²⁵I or ¹⁸F.

Prior to step a) of the present method of preparing a compound of Formula VIII, the ligand (L) portion contains an appropriate reactive moiety for covalently bonding to the reactant having the structure Formula I. In this aspect, L has the following structure: 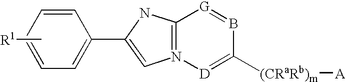
wherein, R^{a}, R^{b}, R¹, m, G, B, and D are as described above, and A is an appropriate group for covalently bonding with Formula I. Examples of appropriate groups include: —OH.

The appropriate ligand portion of Formula VIII compounds can be prepared according to methods fully disclosed in U.S. Pat. No. 6,696,039, herein incorporated by reference in its entirety.

In another embodiment, the invention is directed to the preparation of compounds of Formula IX: 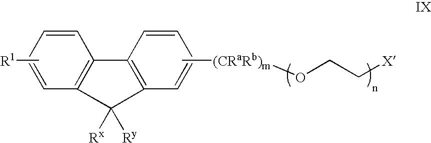
or a pharmaceutically acceptable salt thereof, wherein R¹ is selected from the group consisting of: hydrogen, C₁₋₄ alkyl, amino(C₂₋₄)alkyl, halo(C₁₋₄)alkyl, C₆₋₁₀ aryl, haloarylalkyl, and —NR^{d}R^{e}, wherein R^{d} and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR⁶ in said ring, where R⁶ is hydrogen or C₁₋₄ alkyl; R^{a} and R^{b}, in each instance, is selected from the group consisting of: hydrogen, C₁₋₄ alkyl, di- or mono (C₁₋₄)alkylamino, amino(C₂₋₄)alkyl, halo(C₁₋₄)alkyl, hydroxy(C₁₋₁₀)alkyl and haloarylalkyl; R^{x} and R^{y}, in each instance, is independently selected from the group consisting of hydrogen, C₁₋₄ alkyl, amino(C₂₋₄)alkyl, halo(C₁₋₄)alkyl, C₆₋₁₀ aryl, haloarylalkyl, and —NR^{d}R^{e}, wherein R^{d} and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR⁶ in said ring, where R⁶ is hydrogen or C₁₋₄ alkyl; X′ is selected from the group consisting of: -Ch, ¹²⁵I, ¹³¹I, ¹²³I, ¹⁸F, ⁷⁶Br, or ⁷⁷Br; m is an integer from 0 to 5; and n is an integer from 1 to 10.

Useful values of m are integers from 0 to 5. Preferably, m is an integer from 0 to 2. More preferably, m is 0 or 1.

Useful values of n are integers from 1 to 10. Preferably, n is an integer from 2 to 5. More preferably, n is 3 or 4.

Useful values of X′ include the chelating moiety and all radiohalogens listed above. Preferably, X′ is ¹²³I, ¹²⁵I or ¹⁸F.

Prior to step a) of the present method of preparing a compound of Formula IX as well as Formula X and XI disclosed below, the ligand (L) contains an appropriate reactive moiety for covalently bonding to the reactant having the structure Formula I. The ligand (L) has one of the following structures, wherein A is as described above: 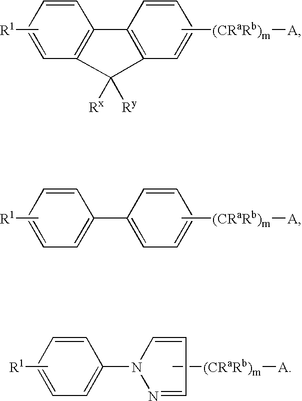
The appropriate ligand portion of Formulae IX, X and XI compounds can be prepared according to methods fully disclosed in published PCT WO 2004/032975 A2, herein incorporated by reference in its entirety.

In another embodiment, the invention is directed to the preparation of compounds of Formula X: 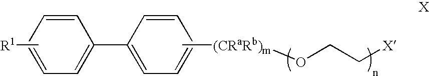
or a pharmaceutically acceptable salt thereof, wherein R¹ is selected from the group consisting of: hydrogen, C₁₋₄ alkyl, amino(C₂₋₄)alkyl, halo(C₁₋₄)alkyl, C₆₋₁₀ aryl, haloarylalkyl, and —NR^{d}R^{e}, wherein R^{d} and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR⁶ in said ring, where R⁶ is hydrogen or C₁₋₄ alkyl; R^{a} and R^{b}, in each instance, is selected from the group consisting of: hydrogen, C₁₋₄ alkyl, di- or mono (C₁₋₄)alkylamino, amino(C₂₋₄)alkyl, halo(C₁₋₄)alkyl, hydroxy(C₁₋₁₀)alkyl and haloarylalkyl; X′ is selected from the group consisting of: -Ch, ¹²⁵I, ¹³¹I, ¹²³I, ¹⁸F, ⁷⁶Br, or ⁷⁷Br; m is an integer from 0 to 5; and n is an integer from 1 to 10.

Useful values of m are integers from 0 to 5. Preferably, m is an integer from 0 to 2. More preferably, m is 0 or 1.

Useful values of n are integers from 1 to 10. Preferably, n is an integer from 2 to 5. More preferably, n is 3 or 4.

Useful values of X′ include the chelating moiety and all radiohalogens listed above. Preferably, X′ is ¹²³I, ¹²⁵I or ¹⁸F.

In another embodiment, the invention is directed to the preparation of compounds of Formula XI: 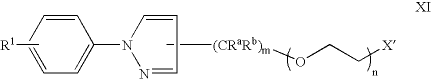
or a pharmaceutically acceptable salt thereof, wherein: R¹ is selected from the group consisting of: hydrogen, C₁₋₄ alkyl, amino(C₂₋₄)alkyl, halo(C₁₋₄)alkyl, C₆₋₁₀ aryl, haloarylalkyl, and —NR^{d}R^{e}, wherein R^{d} and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR⁶ in said ring, where R⁶ is hydrogen or C₁₋₄ alkyl; R^{a} and R^{b}, in each instance, is selected from the group consisting of: hydrogen, C₁₋₄ alkyl, di- or mono (C₁₋₄)alkylamino, amino(C₂₋₄)alkyl, halo(C₁₋₄)alkyl, hydroxy(C₁₋₁₀)alkyl and haloarylalkyl; X′ is selected from the group consisting of: -Ch, ¹²⁵I, ¹³¹I, ¹²³I, ¹⁸F, ⁷⁶Br, or ⁷⁷Br; m is an integer from 0 to 5; and n is an integer from 1 to 10.

Useful values of m are integers from 0 to 5. Preferably, m is an integer from 0 to 2. More preferably, m is 0 or 1.

Useful values of n are integers from 1 to 10. Preferably, n is an integer from 2 to 5. More preferably, n is 3 or 4.

Useful values of X′ include the chelating moiety and all radiohalogens listed above. Preferably, X′ is ¹²³I, ¹²⁵I or ¹⁸F.

A compound of Formula XII, 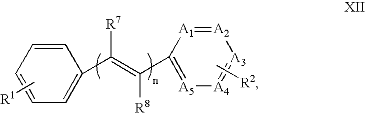
or a pharmaceutically acceptable salt thereof; wherein, n is an integer from one to six; at least one, no more than three, of A₁, A₂, A₃, A₄ and A₅ is N, the others are —CH or —CR² as permitted; R¹ is hydroxy or NR^{a}R^{b}(CH₂)ₚ—, wherein p is an integer from 0 to 5, and R^{a} and R^{b} are independently hydrogen, C₁₋₄ alkyl or (CH₂)_{d}X, where X is halogen, and d is an integer from 1 to 4,

R² is selected from the group consisting of:

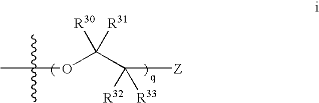

wherein q is an integer from 1 to 10; Z is selected from the group consisting of halogen, halogen substituted benzoyloxy, halogen substituted benzyloxy, halogen substituted phenyl(C₁₋₄)alkyl, halogen substituted aryloxy, and a halogen substituted C₆₋₁₀ aryl; and R³⁰, R³¹, R³² and R³³ are in each instance independently selected from the group consisting of hydrogen, hydroxy, C₁₋₄ alkoxy, C₁₋₄ alkyl, and hydroxy(C₁₋₄)alkyl;

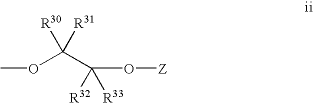

wherein Z, R³⁰, R³¹, R³² and R³³ are as described above,

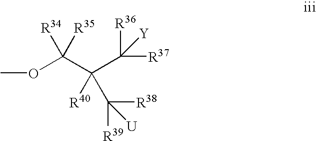

wherein Y is selected from the group consisting of halogen, halogen substituted benzoyloxy, halogen substituted phenyl(C₁₋₄)alkyl, halogen substituted aryloxy, and halogen substituted C₆₋₁₀ aryl;

U is selected from the group consisting of hydrogen, hydroxy, halogen, halogen substituted benzoyloxy, halogen substituted phenyl(C₁₋₄)alkyl, halogen substituted aryloxy, and halogen substituted C₆₋₁₀ aryl; and

R³⁴, R³⁵, R³⁶, R³⁷, R³⁸, R³⁹ and R⁴⁰ are in each instance independently selected from the group consisting of hydrogen, halogen, hydroxy, C₁₋₄ alkoxy, C₁₋₄ alkyl, and hydroxy(C₁₋₄)alkyl;

iv. NR′R″, wherein at least one of R′ and R″ is (CH₂)_{d}X, where X is halogen, preferably F or ¹⁸F, and d is an integer from 1 to 4; the other of R′ and R″ is selected from the group consisting of hydrogen, C₁₋₄ alkyl, halo(C₁₋₄)alkyl, and hydroxy(C₁₋₄)alkyl;

v. NR′R″—(C₁₋₄)alkyl, wherein at least one of R′ and R″ is (CH₂)_{d}X, where X is halogen, preferably F or ¹⁸F, and d is an integer from 1 to 4; the other of R′ and R″ is selected from the group consisting of hydrogen, C₁₋₄ alkyl, halo(C₁₋₄)alkyl, and hydroxy(C₁₋₄)alkyl;

vi. halo(C₁₋₄)alkyl; and

vii. an ether (R—O—R) having the following structure: [halo(C₁₋₄)alkyl-O—(C₁₋₄)alkyl]-; and

R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl.

Preferred compounds include those where the halogen, in one or more occurrence on the structure, is a radiolabeled halogen. Also preferred are compounds wherein the halogen is selected from the group consisting of I, ¹²³I, ¹²⁵I, ¹³¹I, Br, ⁷⁶Br, ⁷⁷Br, F or ¹⁸F. Especially preferred compounds are those that contain ¹⁸F.

Useful values of R¹ are listed above. Useful values of p include integers from 0 to 5. Preferably, p is 0, 1 or 2. Most preferably, p is 0 such that R¹ represents NR^{a}R^{b}. In preferred embodiments, R¹ is either in the meta or para position relative to the respective bridge. A preferred value of R¹ is NR^{a}R^{b}, wherein R^{a} and R^{b} are independently hydrogen or C₁₋₄ alkyl. In this embodiment, it is preferable that the C₁₋₄ alkyl is methyl. Most preferably, both R^{a} and R^{b} are methyl.

Useful values of n include integers from 1 to 6. Preferably, the value of n is from 1 to 4. Most preferably, the value of n is from 1 to 3.

Useful values of R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl. The value of n determines the number of R⁷ and R⁸ group(s) present in the compound. If present more than once in a particular compound, in each instance of R⁷ and R⁸ the value can be different from any other value of R⁷ and R⁸. In preferred embodiments, R⁷ and R⁸ are each hydrogen in every instance.

Useful values of R² include substructures i, ii, iii, iv, v, vi and vii, as depicted above. In preferred embodiments of Formula I, R² is either in the meta or para position relative to the respective bridge. Preferably, R² is substructure i or iii. In these embodiments, useful values of q include integers from one to ten. Preferably, in a compound where R² is i, q is an integer from 1 to 5. Most preferably, q is 3 or 4. In substructure i, useful values of R³⁰, R³¹, R³² and R³³ independently include hydrogen, hydroxy, C₁₋₄ alkoxy, C₁₋₄ alkyl, and hydroxy(C₁₋₄)alkyl. Preferred compounds include those where one or more of R³⁰, R³¹, R³² and R³³ are hydrogen. More preferred compounds include those where each of R³⁰, R³¹, R³² and R³³ is hydrogen.

In substructure ii, useful values of Y, U and R³⁴, R³⁵, R³⁶, R³⁷, R³⁸, R³⁹ and R⁴⁰ are described above. Preferred compounds include those where U is hydroxy.

Useful compounds include those compounds where at least one, no more than three, of A₁, A₂, A₃, A₄ and A₅ is N, and the others are —CH or —CR² as permitted. It is preferred that if only one, no more than three, of A₁, A₂, A₃, A₄ and A₅ is N, that it is A₄.

Another aspect of the present invention is directed to compounds of Formulae IV, VI, VII, VIII, IX, X, XI and XII, and compositions comprising the compounds.

Another aspect of the present invention is directed to compounds of Formulae IV, VI, VII, VIII, IX, X, XI and XII prepared according to the method described herein.

Another aspect of the present invention is directed to a method of imaging amyloid deposits comprising, a) administering to a mammal an amount of an imaging agent, said agent comprising a Ligand (L) that binds amyloid deposits covalently attached to a moiety (X′), and having the following Formula IV, 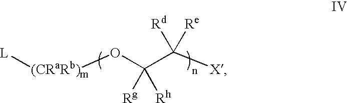
wherein, X′ is selected from the group consisting of hydrogen, hydroxy, C₁₋₄ alkoxy, halogen, radiohalogen, 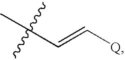
wherein Q is a halogen or radiohalogen, and a chelating moiety bound to a radio-metal; R^{a}, R^{b}, R^{d}, R^{e}, R^{g} and R^{h} are, in each instance, independently selected from the group consisting of hydrogen, hydroxy, C₁₋₄ alkoxy, C₁₋₄ alkyl, and hydroxy(C₁₋₄)alkyl; m is an integer from 0 to 5; and n is an integer from 1 to 10;
b) allowing sufficient time for said agent to become associated with one or more amyloid deposits in said mammal; and
c) detecting said agent associated with said one or more amyloid deposits;
   provided,

that one of X′ or Q either contains a radiohalogen or radiometal as permitted, or (L) is covalently bonded to a radiohalogen; and

that in Formula IV, when m is zero, L is other than: 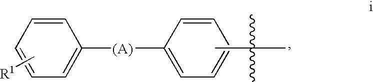
or a pharmaceutically acceptable salt thereof, wherein:

A is selected from the group consisting of: 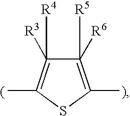
wherein R³, R⁴, R⁵ and R⁶ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl;

and 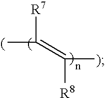
wherein n is an integer between 1 and 6; and R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl;

R¹ is selected from the group consisting of:
a. NR^{a′}R^{b′}, wherein R^{a′} and R^{b′} are independently hydrogen, C₁₋₄ alkyl or (CH₂)_{d}X, where X is halogen, and d is an integer between 1 and 4,
b. hydroxy,
c. C₁₋₄alkoxy, and
d. hydroxy(C₁₋₄)alkyl.

Preferred values of L have the following structures, L1, L1′, L2, L2′, L3, L3′, L4, L5, L6, L6′, L7, L7′, L8 and L9, described below where 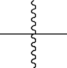
denotes the point of attachment of L at the —(CR^{a}R^{b})ₘ— group if present, or if m is 0, the point of attachment of L with the EG or PEG moiety of Formula IV: 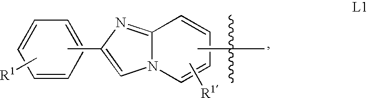
wherein, R¹ and R^{1′}, are in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; 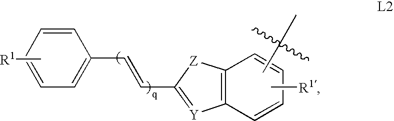
wherein R¹ and R^{1′}, are in each instance, independently selected from the group consisting of: hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; q is an integer from 0 to 3; Z is O, S or N; Y is N or —CH; in this embodiment, it is preferable that q is 0 or 1; 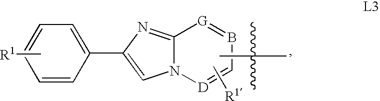
wherein, G, B and D are CH or N, provided that at least one no more than two of G, B and D is N; and R¹ and R^{1′}, are in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; 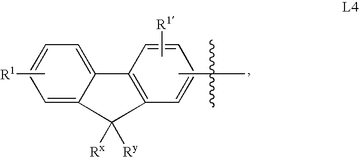
wherein, R¹ and R^{1′} are, in each instance, independently selected from the group consisting of: hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₂₋₄)alkyl, C₆₋₁₀ aryl, haloarylalkyl, and —NR^{d}R^{e}, wherein R^{d}and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR⁶ in said ring, where R⁶ is hydrogen or C₁₋₄ alkyl; R^{x} and R^{y}, in each instance, is independently selected from the group consisting of hydrogen, C₁₋₄ alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; 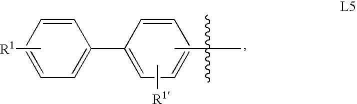
wherein, R¹ and R^{1′} are, in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; 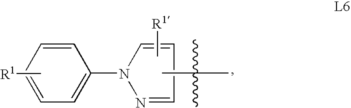
wherein, R¹ and R^{1′} are, in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; 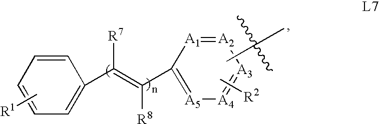
wherein, n is an integer from one to six; at least one, no more than three, of A₁, A₂, A₃, A₄ and A₅ is N, the others are —CH or —CR₂ as permitted; R¹ and R², in each instance, are independently selected from the group consisting of hydrogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and NR^{a′}R^{b′}(CH₂)ₚ—, wherein p is an integer from 0 to 5, and R^{a′} and R^{b′}, in each instance, is independently selected from the group consisting of: hydrogen, C₁₋₄ alkyl and halo(C₁₋₄)alkyl, or R^{a′} and R^{b′} are taken together with the nitrogen to which they are attached to form a 5- to 7-member heterocyclic ring optionally having O, S or NR⁶ in said ring, where R⁶ is hydrogen or C₁₋₄ alkyl are independently hydrogen, C₁₋₄ alkyl or (CH₂)_{d}X, where X is halogen, and d is an integer from 1 to 4, and R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl; 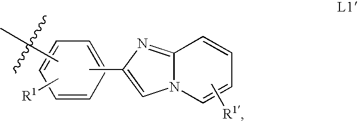
wherein, R¹ and R^{1′}, are in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; 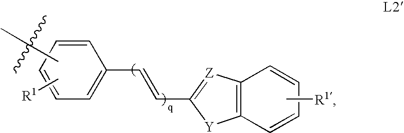
wherein, R¹ and R^{1′}, are in each instance, independently selected from the group consisting of: hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; q is an integer from 0 to 3; Z is O, S or N; and Y is N or —CH; 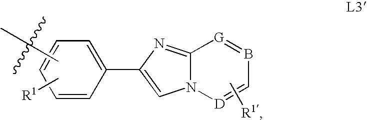
wherein, G, B and D are CH or N, provided that at least one no more than two of G, B and D is N; and R¹ and R^{1′}, are in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; 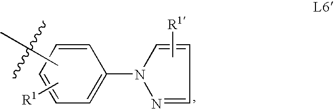
wherein, R¹ and R^{1′} are, in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl; 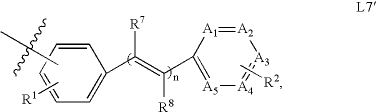
wherein, n is an integer from one to six; at least one, no more than three, of A₁, A₂, A₃, A₄ and A₅ is N, the others are —CH or —CR² as permitted; R¹ and R², in each instance, are independently selected from the group consisting of hydrogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and NR^{a′}R^{b′}(CH₂)ₚ—, wherein p is an integer from 0 to 5, and R^{a′} and R^{b′}, in each instance, is independently selected from the group consisting of: hydrogen, C₁₋₄ alkyl and halo(C₁₋₄)alkyl, or R^{a′} and R^{b′} are taken together with the nitrogen to which they are attached to form a 5- to 7-member heterocyclic ring optionally having O, S or NR⁶ in said ring, where R⁶ is hydrogen or C₁₋₄ alkyl are independently hydrogen, C₁₋₄ alkyl or (CH₂)_{d}X, where X is halogen, and d is an integer from 1 to 4, and R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl; 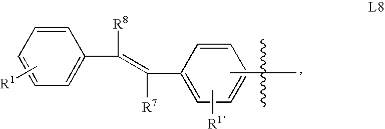
wherein, n is an integer from one to six; R¹ and R^{1′}, in each instance, are independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and NR^{a′}R^{b′}(CH₂)ₚ—, wherein p is an integer from 0 to 5, and R^{a′} and R^{b ′}, in each instance, is independently selected from the group consisting of: hydrogen, C₁₋₄ alkyl and halo(C₁₋₄)alkyl, or R^{a′} and R^{b′} are taken together with the nitrogen to which they are attached to form a 5- to 7-member heterocyclic ring optionally having O, S or NR⁶ in said ring, where R⁶ is hydrogen or C₁₋₄ alkyl are independently hydrogen, C₁₋₄ alkyl or (CH₂)_{d}X, where X is halogen, and d is an integer from 1 to 4, and R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl; 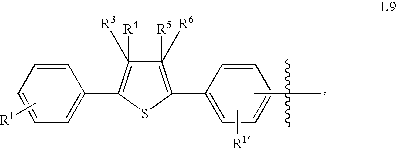
wherein, n is an integer from one to six; R¹ and R^{1′}, in each instance, are independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and NR^{a′}R^{b′}(CH₂)ₚ—, wherein p is an integer from 0 to 5, and R^{a′} and R^{b′}, in each instance, is independently selected from the group consisting of: hydrogen, C₁₋₄ alkyl and halo(C₁₋₄)alkyl, or R^{a′} and R^{b′} are taken together with the nitrogen to which they are attached to form a 5- to 7-member heterocyclic ring optionally having O, S or NR⁶ in said ring, where R⁶ is hydrogen or C₁₋₄ alkyl are independently hydrogen, C₁₋₄ alkyl or (CH₂)_{d}X, where X is halogen, and d is an integer from 1 to 4, and R³, R⁴, R⁵ and R⁶ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl.

In all the above embodiments, it is preferable that one of R¹ and R^{1′} is selected from the group consisting of hydrogen, halogen, radiohalogen, and NR^{a′}R^{b′}(CH₂)ₚ—, wherein p is an integer from 0 to 5, and R^{a′} and R^{b′}, in each instance, is independently selected from the group consisting of: hydrogen and C₁₋₄ alkyl.

Where applicable, preferred values of R⁷ and R⁸ are independently hydrogen and C₁₋₄ alkyl.

Preferably, the radiohalogen is selected from the group consisting of ¹⁸F, ¹³¹I, ¹²⁵I, ¹²³I, ¹²⁴I, ⁷⁷Br and ⁷⁶Br. Most preferably, the radiohalogen is ¹⁸F.

When the radiolabel is a radiometal, it can be a radioisotope of Technetium, Copper, Indium, or Gallium. Preferably, the radiometal is 99m-Tc. Preferably, the chelating moiety is a N₂S₂ type chelating agent as described more fully herein.

The above method can further comprise measuring the distribution of the radiolabeled compound by preferably using either positron emission tomography (PET) or single photon emission tomography (SPECT).

In another aspect, the present invention is directed to a method of imaging amyloid deposits comprising: a) administering to a mammal a first ligand capable of binding amyloid deposits in the brain; b) allowing sufficient time for said first ligand to become associated with one or more amyloid deposits in said mammal; and c) detecting said first ligand associated with said amyloid deposits; the improvement comprising covalently attaching to said first ligand a group to provide a second ligand having attached thereto a radiolabel suitable for imaging without a substantial increase in the lipophilicity of said first ligand, said group having the following structure: 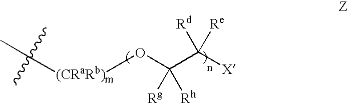
wherein R^{a}, R^{b}, R^{d}, R^{e}, R^{g}, R^{h}, m, n are as described above, and X′ is selected from the group consisting of a radiohalogen, 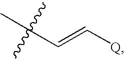
wherein Q is a radiohalogen, and a chelating moiety bound to a radio-metal; provided,

that if m is zero, said first ligand is other than: 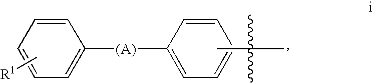
or a pharmaceutically acceptable salt thereof, wherein:

A is selected from the group consisting of: 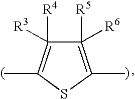
wherein R³, R⁴, R⁵ and R⁶ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl;

and 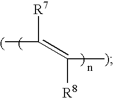
wherein n is an integer between 1 and 6; and R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl;

R¹ is selected from the group consisting of:
a. NR^{a′}R^{b′}, wherein R^{a′} and R^{b′} are independently hydrogen, C₁₋₄ alkyl or (CH₂)_{d}X, where X is halogen, and d is an integer between 1 and 4,
b. hydroxy,
c. C₁₋₄ alkoxy, and
d. hydroxy(C₁₋₄)alkyl.

In another aspect, the present invention is directed to a pharmaceutical composition comprising, (a) a compound capable of binding amyloid deposits, having a relatively low rate of transfer across a blood-brain barrier and having a core structure L1, L1′, L2, L2′, L3, L3′, L4, L5, L6, L6′, L7, L7′, L8 or L9 as described herein, the improvement comprising covalently attaching a group (Z) to said compound to provide imaging compounds having increased rates of transfer across a blood-brain barrier, wherein (Z) has the following formula: 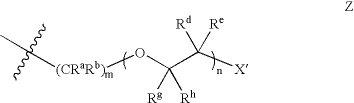
wherein R^{a}, R^{b}, R^{d}, R^{e}, R^{g}, R^{h}, m, n and X′ are as described above; and (b) pharmaceutically acceptable diluents or excipients.

It is also to be understood that the present invention is considered to include stereoisomers as well as optical isomers, e.g. mixtures of enantiomers as well as individual enantiomers and diastereomers, which arise as a consequence of structural asymmetry in selected compounds of the present series.

The compounds disclosed herein may also be solvated, especially hydrated. Hydration may occur during manufacturing of the compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

When any variable occurs more than one time in any constituent or in compounds described herein, its definition on each occurrence is independent of its definition at every other occurrence. Also combinations of substituents and/or variables are permissible only if such combinations result in stable compounds.

The present invention further relates to a method of preparing a technetium-99m complex according to the present invention by reacting technetium-99m in the form of a pertechnetate in the presence of a reducing agent and optionally a suitable chelator with an appropriate Ch-containing compound.

The reducing agent serves to reduce the Tc-99m pertechnetate which is eluted from a molybdenum-technetium generator in a physiological saline solution. Suitable reducing agents are, for example, dithionite, formamidine sulphinic acid, diaminoethane disulphinate or suitable metallic reducing agents such as Sn(II), Fe(II), Cu(I), Ti(III) or Sb(III). Sn(II) has proven to be particularly suitable.

For the above-mentioned complex-forming reaction, technetium-99m is reacted with an appropriate compound of the invention as a salt or in the form of technetium bound to comparatively weak chelators. In the latter case the desired technetium-99m complex is formed by ligand exchange. Examples of suitable chelators for the radionuclide are dicarboxylic acids, such as oxalic acid, malonic acid, succinic acid, maleic acid, orthophtalic acid, malic acid, lactic acid, tartaric acid, citric acid, ascorbic acid, salicylic acid or derivatives of these acids; phosphorus compounds such as pyrophosphates; or enolates. Citric acid, tartaric acid, ascorbic acid, glucoheptonic acid or a derivative thereof are particularly suitable chelators for this purpose, because a chelate of technetium-99m with one of these chelators undergoes the desired ligand exchange particularly easily.

The most commonly used procedure for preparing [TcvO]⁺³N₂S₂ complexes is based on stannous (II) chloride reduction of [^{99m}Tc]pertechnetate, the common starting material. The labeling procedure normally relies on a Tc-99m ligand exchange reaction between Tc-99m (Sn)-glucoheptonate and the N₂S₂ ligand. Preparation of stannous (II) chloride and preserving it in a consistent stannous (II) form is critically important for the success of the labeling reaction. To stabilize the air-sensitive stannous ion it is a common practice in nuclear medicine to use a lyophilized kit, in which the stannous ion is in a lyophilized powder form mixed with an excess amount of glucoheptonate under an inert gas like nitrogen or argon. The preparation of the lyophilized stannous chloride/sodium glucoheptonate kits ensures that the labeling reaction is reproducible and predictable. The N₂S₂ ligands are usually air-sensitive (thiols are easily oxidized by air) and there are subsequent reactions which lead to decomposition of the ligands. The most convenient and predictable method to preserve the ligands is to produce lyophilized kits containing 100-500 μg of the ligands under argon or nitrogen.

The term “alkyl” as employed herein by itself or as part of another group refers to both straight and branched chain radicals of up to 8 carbons, preferably 6 carbons, more preferably 4 carbons, such as methyl, ethyl, propyl, isopropyl, butyl, t-butyl, and isobutyl.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

The term “monoalkylamine” as employed herein by itself or as part of another group refers to an amino group which is substituted with one alkyl group as defined above.

The term “dialkylamine” as employed herein by itself or as part of another group refers to an amino group which is substituted with two alkyl groups as defined above.

The term “halo” employed herein by itself or as part of another group refers to chlorine, bromine, fluorine or iodine.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl.

The term “heterocycle” or “heterocyclic ring”, as used herein except where noted, represents a stable 5- to 7-membered mono-heterocyclic ring system which may be saturated or unsaturated, and which consists of carbon atoms and from one to three heteroatoms selected from the group consisting of N, O, and S, and wherein the nitrogen and sulfur heteroatom may optionally be oxidized. Especially useful are rings contain one nitrogen combined with one oxygen or sulfur, or two nitrogen heteroatoms. Examples of such heterocyclic groups include piperidinyl, pyrrolyl, pyrrolidinyl, imidazolyl, imidazlinyl, imidazolidinyl, pyridyl, pyrazinyl, pyrimidinyl, oxazolyl, oxazolidinyl, isoxazolyl, isoxazolidinyl, thiazolyl, thiazolidinyl, isothiazolyl, homopiperidinyl, homopiperazinyl, pyridazinyl, pyrazolyl, and pyrazolidinyl, most preferably thiamorpholinyl, piperazinyl, and morpholinyl.

The term “heteroatom” is used herein to mean an oxygen atom (“O”), a sulfur atom (“S”) or a nitrogen atom (“N”). It will be recognized that when the heteroatom is nitrogen, it may form an NR^{d}R^{e} moiety, wherein R^{d} and R^{e} are, independently from one another, hydrogen or C₁₋₄ alkyl, C₂₋₄ aminoalkyl, C₁₋₄ halo alkyl, halo benzyl, or R^{d} and R^{e} are taken together to form a 5- to 7-member heterocyclic ring optionally having O, S or NR^{c} in said ring, where R^{c} is hydrogen or C₁₋₄ alkyl.

The present invention is directed to a methods of preparing compounds of the above Formula V, VI, VII, VIII, IX, X, XI or XII. One of the major advantages of our FPEG approach is incorporation of the fluoro tag at the end of a polyethylene glycol chain. The preparation of these compounds is readily achieved in a relatively simple and straightforward manor. Synthesis of core compounds 2 and 4 and polyethylene glycol precursors was accomplished following literature procedures with minor modifications (20, 25). Compound 3′, the N′,N″-dimethylamino derivative of 3 was prepared from 6-hydroxy-2-methylbenzoxazole followed by in situ trimethylsilyl protection of the phenolic OH, deprotonation and condensation with N,N′-dimethylaminobenzaldehyde as described during the synthesis of similar compounds by Schreiner and co-workers (Scheme 1) (28). Conjugation of the free phenolic hydroxyl groups to compounds 3′ and 4 with various oligoethylene glycol precursors was accomplished under microwave irradiation in good yields (Scheme 1 A and B). Utilizing the same methodology the radiofluorination precursors can be generated quickly and efficiently, conveniently allowing the radioactive fluoride to be added in the last step of the synthesis. Preparation of the mesylate precursor was generated following synthesis of the hydroxyl derivative using a similar microwave procedure (Scheme 2C). It was important to also prepare the hydroxy derivatives as it competes for binding to beta amyloid plaques and is the major by-product during radiolabeling. The synthetic versatility of the strategy was further demonstrated with conjugates of compound 2 wherein FPEG was conjugated to 2 via a copper catalyzed coupling reaction with the aryl iodide and corresponding fluoro/hydroxy PEG derivative (Scheme 3). The desired FPEG derivatives were prepared in moderate to good yield. This approach has proven effective, but is not universally appropriate. For instance, if the pegylated ligand exhibits lower affinity for the target amyloid or is too lipophilic or hydrophilic for brain and CNS imaging.

Radiolabeling with ¹⁸F was performed on precursors 10a-c (Scheme 3) and 11 to generate [¹⁸F]5a-c and [¹⁸F]8b respectively. ¹⁸F labeling of compounds 12a-e was not pursued due to their poor in vitro binding affinities (Table 1) and compound 8b was chosen due to the very promising in vitro results. Radiolabeled [¹⁸F]8b was prepared from the mesylate precursor in moderate radiochemical yield (23%) but unfortunately could not be prepared in good radiochemical purity. The formation of a second peak was evident within minutes of labeling. These results are consistent with those found by Shimadzu et. al. during their labeling of a similar substrate. They attribute the formation of a second peak to the facile formation of E and Z isomers (30). As a result, we focused our remaining labeling studies on compounds 5a-d (PIB core), which had also shown promising in vitro results.

The use of mesylate precursors for radiofluorination chemistry has been used for many years (18), however the use of mesylate precursors for radiolabeling FPEG conjugates has never been optimized. Based on the promising biological results compound 5a was chosen for some optimization studies, examining the effects of precursor mass, temperature, reaction time and purification sep-pak strategies using traditional oil bath methods.

Initially, using 1 mg of precursor 10a dissolved in 250 μL of dimethyl sulfoxide, the reaction temperature was varied from 75° C. to 120° C. using standard oil bath heating for 4 minutes. Deprotection of the BOC protecting group was then achieved by adding 10% HCl and heating for 10 minutes. Water was then added (2 mL) and the solution loaded onto an Oasis HLB sep-pak cartridge. Following washing with water, the crude labeled product was eluted with 2 mL of acetonitrile and injected onto the HPLC. Labeling yields were highest at 120° C. (Table 2). Next, the amount of precursor (10a) was varied from 0.5 mg to 6 mg with oil bath heating at 120° C. for 4 minutes. BOC deprotection was then accomplished as described above leading to radiochemical yields ranging from 30-50%, with the highest between 1 and 3 mg. The final study performed with traditional oil bath heating evaluated the effect of increasing the reaction times from 4 minutes to 16 minutes using 1 mg of precursor and heating at 120° C. We found that reaction times from 8-16 minute all led to high radiochemical yields of greater than 59%. The radiochemical purity for all reactions was greater than 98%.

From these studies it is evident that traditional oil bath strategies can prepare radiolabeled [¹⁸F]5a conjugates in good radiochemical yields (60-64%). The optimized conditions are 1-3 mg of precursor heated at 120° C. for 12 minutes, followed by the standard BOC deprotection.

Incorporating a radioactive fluoride atom is typically accomplished using either electrophilic or nucleophilic conditions (17-19). Fluoride nucleophilic displacement reactions are advantageous as they often result in higher yields, higher specific activities and the fluoride can be produced more readily (18, 19). [¹⁸F] fluoride can be added via an SN2 type reaction with good leaving groups such as either the mesylate or tosylate precursor. The most commonly used method to append a fluorine atom involves adding a fluoroethyl or fluoropropyl group to the target compound. However, when these short fluoro alkyl chains were added to the core structures the results were sometimes not promising. This is often due to an increase in lipophilicity; the resulting ¹⁸F labeled agents tend to have a higher non-specific binding and a lower specific binding to the Aβ aggregates. To circumvent these undesirable effects we have exploited a novel approach by using fluoro-pegylation (FPEG) of the core structures for ¹⁸F labeling of stilbene derivatives (20).

Pegylation using high MW (10,000-20,000) is a common approach for changing in vivo pharmacokinetics of various biologically interesting proteins or peptides, through which the in vivo stability and pharmacokinetics can be improved leading to better therapeutics (21, 22). Recently, a pegylation technique has also been applied to modify pharmacokinetic properties of radiopharmaceuticals (23, 24). Conjugating PEG macromolecules to labeled peptides may be efficacious in changing biodistribution in vivo and leading to improvements in specific localization of agents targeting peripheral tissues. However, it will be ineffective to use macromolecular PEG conjugated radiopharmaceuticals as imaging agents for the brain due to limitation of such macromolecules to cross the blood-brain barrier. We have adopted a novel approach by adding a short length of FPEG (n=2-5) and capping the end of the ethylene glycol chain with a fluorine atom (20).

The compounds of this invention can be prepared by reactions described in the following schemes. Scheme 1 depicts a synthetic route for preparing FPEG PIB (5a-d) and BF (8a-d) conjugates (compounds of Formula VII).

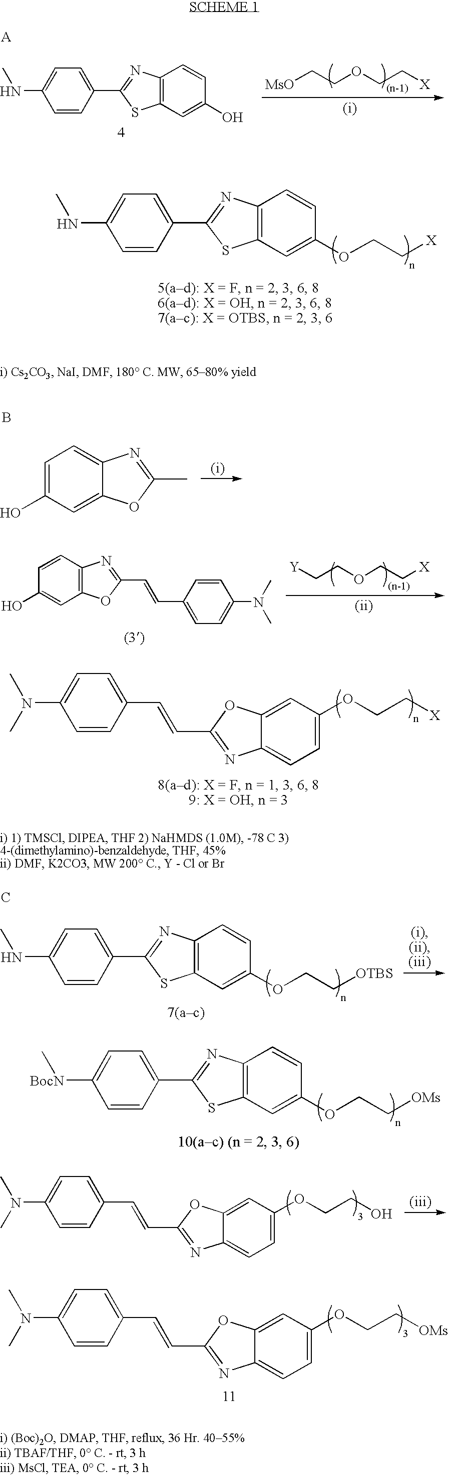

The following Scheme 2 depicts a synthetic route for preparing FPEG-IMPY conjugates (compounds of Formula VI).

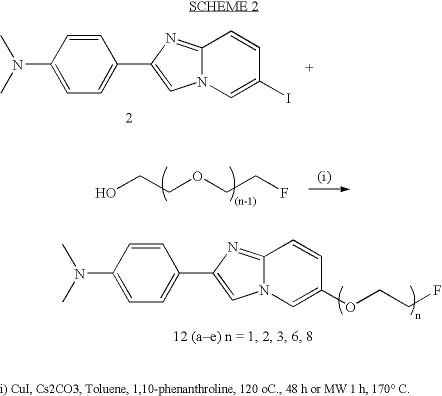

The following Scheme 3 depicts the ¹⁸F radiolabeling of 10a-c.

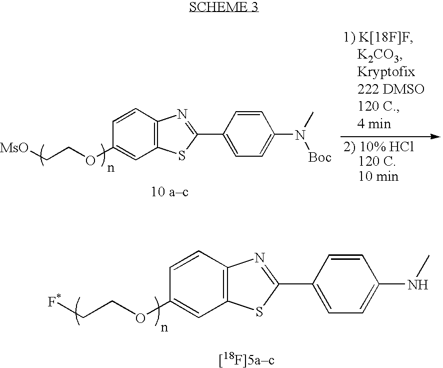

The following Scheme 4 depicts a synthetic route for preparing compounds of Formula I.

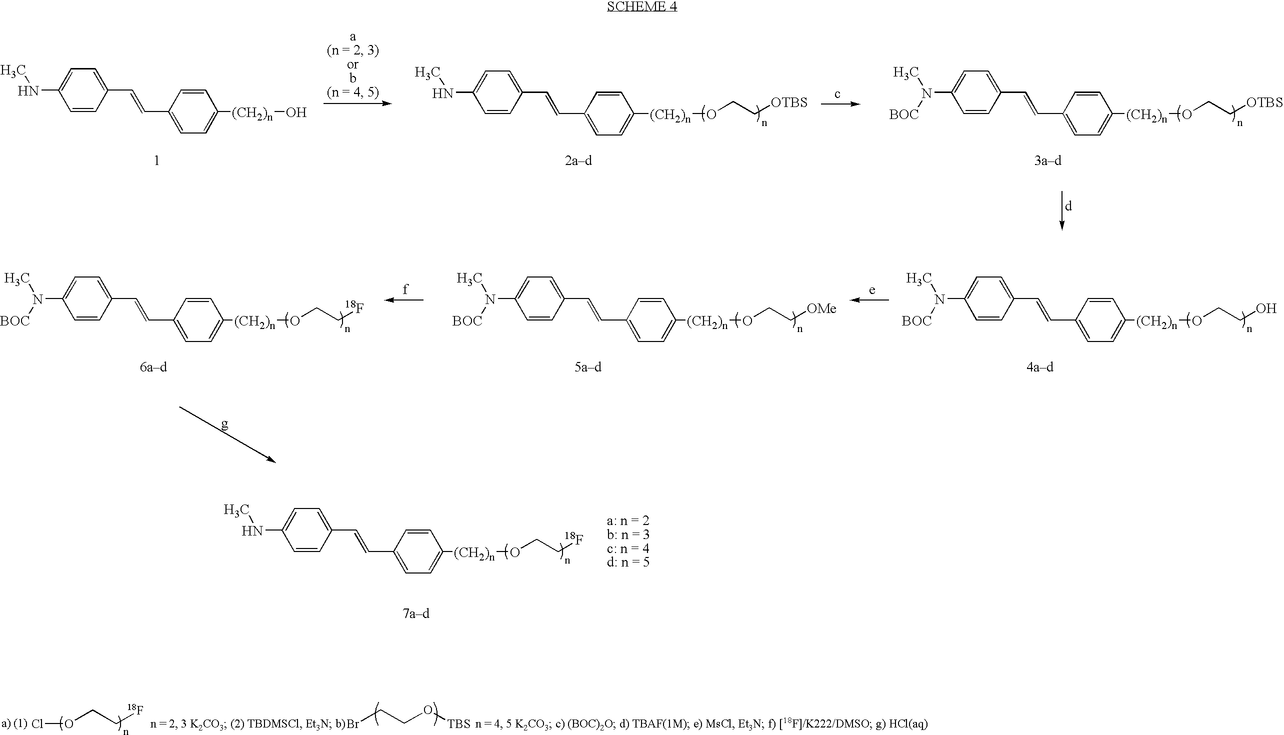

The following Scheme 5 depicts a synthetic route for preparing a compound of Formula IV, wherein L is L7.

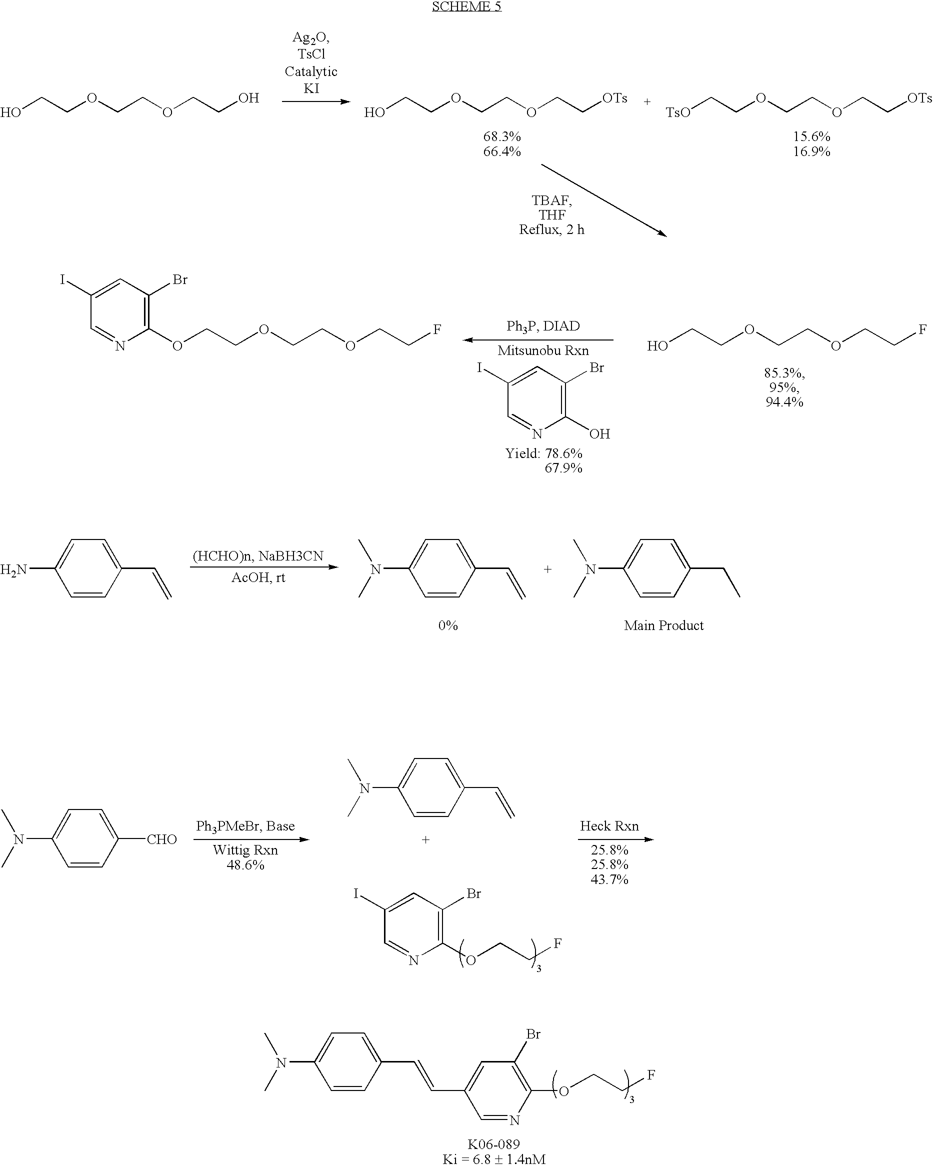

It is also to be understood that the present invention is considered to include stereoisomers as well as optical isomers, e.g. mixtures of enantiomers as well as individual enantiomers and diastereomers, which arise as a consequence of structural asymmetry in selected compounds of the present invention.

The compounds of the present invention may also be solvated, especially hydrated. Hydration may occur during manufacturing of the, compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

When any variable occurs more than one time in any constituent or in and structure or Formulae herein, its definition on each occurrence is independent of its definition at every other occurrence. Also combinations of substituents and/or variables are permissible only if such combinations result in stable compounds.

When the compounds of this invention are to be used as imaging agents, they must be labeled with suitable radioactive halogen isotopes. Although ¹²⁵I-isotopes are useful for laboratory testing, they will generally not be useful for actual diagnostic purposes because of the relatively long half-life (60 days) and low gamma-emission (30-65 Kev) of ¹²⁵I. The isotope ¹²³I has a half life of thirteen hours and gamma energy of 159 KeV, and it is therefore expected that labeling of ligands to be used for diagnostic purposes would be with this isotope or ¹⁸F (half life of 2 hours). Other isotopes which may be used include ¹³¹I. Suitable bromine isotopes include ⁷⁷Br and ⁷⁶Br.

Tc 99m complexes can be prepared as follows. A small amount of non-radiolabeled compound (1-2 mg) is dissolved in 100 μL EtOH and mixed with 200 μL HCl (1 N) and 1 mL Sn glucoheptonate solution (containing 8-32 μg SnCl2 and 80 320 μg Na glucoheptonate, pH 6.67) and 50 μL EDTA solution (0.1 N). [99mTc]Pertechnetate (100-200 μL; ranging from 2-20 mCi) saline solution are then added. The reaction is heated for 30 min at 100° C., then cooled to room temperature. The reaction mixture is analyzed on TLC (EtOH:conc. NH₃ 9:1) for product formation and purity check. The mixture can be neutralized with phosphate buffer to pH 5.0.

The present invention further relates to a method of preparing a technetium-99m complex according to the present invention by reacting technetium-99m in the form of a pertechnetate in the presence of a reducing agent and optionally a suitable chelator with an appropriate Ch-containing compound.

The reducing agent serves to reduce the Tc-99m pertechnetate which is eluted from a molybdenum-technetium generator in a physiological saline solution. Suitable reducing agents are, for example, dithionite, formamidine sulphinic acid, diaminoethane disulphinate or suitable metallic reducing agents such as Sn(II), Fe(II), Cu(I), Ti(III) or Sb(III). Sn(II) has proven to be particularly suitable.

For the above-mentioned complex-forming reaction, technetium-99m is reacted with an appropriate compound of the invention as a salt or in the form of technetium bound to comparatively weak chelators. In the latter case the desired technetium-99m complex is formed by ligand exchange. Examples of suitable chelators for the radionuclide are dicarboxylic acids, such as oxalic acid, malonic acid, succinic acid, maleic acid, orthophtalic acid, malic acid, lactic acid, tartaric acid, citric acid, ascorbic acid, glucoheptonic acid, salicylic acid or derivatives of these acids; phosphorus compounds such as pyrophosphates; or enolates. Citric acid, tartaric acid, ascorbic acid, glucoheptonic acid or a derivative thereof are particularly suitable chelators for this purpose, because a chelate of technetium-99m with one of these chelators undergoes the desired ligand exchange particularly easily.

The most commonly used procedure for preparing [TcvO]⁺³N₂S₂ complexes is based on stannous (II) chloride reduction of [99mTc]pertechnetate, the common starting material. The labeling procedure normally relies on a Tc 99m ligand exchange reaction between Tc 99m (Sn) glucoheptonate and the N₂S₂ ligand. Preparation of stannous (II) chloride and preserving it in a consistent stannous (II) form is critically important for the success of the labeling reaction. To stabilize the air sensitive stannous ion it is a common practice in nuclear medicine to use a lyophilized kit, in which the stannous ion is in a lyophilized powder form mixed with an excess amount of glucoheptonate under an inert gas like nitrogen or argon. The preparation of the lyophilized stannous chloride/sodium glucoheptonate kits ensures that the labeling reaction is reproducible and predictable. The N₂S₂ ligands are usually air sensitive (thiols are easily oxidized by air) and there are subsequent reactions which lead to decomposition of the ligands. The most convenient and predictable method to preserve the ligands is to produce lyophilized kits containing 100-500 μg of the ligands under argon or nitrogen.

The radiohalogenated compounds of this invention lend themselves easily to formation from materials which could be provided to users in kits. Kits for forming the imaging agents can contain, for example, a vial containing a physiologically suitable solution of an intermediate of a radiolabeled compound of the present invention in a concentration and at a pH suitable for optimal complexing conditions. The user would add to the vial an appropriate quantity of the radioisotope, e.g., Na¹²³I, and an oxidant, such as hydrogen peroxide. The resulting labeled ligand may then be administered intravenously to a patient, and receptors in the brain imaged by means of measuring the gamma ray or photo emissions therefrom.

When the compounds of this invention are to be used as imaging agents, they must be labeled with suitable radioactive halogen isotopes. Although ¹²⁵I-isotopes are useful for laboratory testing, they will generally not be useful for actual diagnostic purposes because of the relatively long half-life (60 days) and low gamma-emission (30-65 Kev) of ¹²⁵I. The isotope ¹²³I has a half life of thirteen hours and gamma energy of 159 KeV, and it is therefore expected that labeling of ligands to be used for diagnostic purposes would be with this isotope, or more preferably ¹⁸F. Other isotopes which may be used include ¹³¹I (half life of 2 hours). Suitable bromine isotopes include ⁷⁷Br and ⁷⁶Br.

The radiohalogenated compounds of this invention lend themselves easily to formation from materials which could be provided to users in kits.

Kits for forming the imaging agents can contain, for example, a vial containing a physiologically suitable solution of an intermediate of Formula IV, wherein L is selected from the group consisting of L1, L1′, L2, L2′, L3, L3′, L4, L5, L6, L6′, L7, L7′, L8 and L9 in a concentration and at a pH suitable for optimal complexing conditions. The user would add to the vial an appropriate quantity of the radioisotope, e.g., Na¹²³I, and an oxidant, such as hydrogen peroxide. The resulting labeled ligand may then be administered intravenously to a patient, and receptors in the brain imaged by means of measuring the gamma ray or photo emissions therefrom.

Since the radiopharmaceutical composition according to the present invention can be prepared easily and simply, the preparation can be carried out readily by the user. Therefore, the present invention also relates to a kit, comprising:

(1) A non-radiolabeled compound of the invention, the compound optionally being in a dry condition; and also optionally having an inert, pharmaceutically acceptable carrier and/or auxiliary substances added thereto; and

(2) a reducing agent and optionally a chelator;

wherein ingredients (1) and (2) may optionally be combined; and further wherein instructions for use with a prescription for carrying out the above-described method by reacting ingredients (1) and (2) with technetium-99m in the form of a pertechnetate solution may be optionally included.

Examples of suitable reducing agents and chelators for the above kit have been listed above. The pertechnetate solution can be obtained by the user from a molybdenum-technetium generator. Such generators are available in a number of institutions that perform radiodiagnostic procedures. As noted above the ingredients (1) and (2) may be combined, provided they are compatible. Such a monocomponent kit, in which the combined ingredients are preferably lyophilized, is excellently suitable to be reacted by the user with the pertechnetate solution in a simple manner.

When desired, the radioactive diagnostic agent may contain any additive such as pH controlling agents (e.g., acids, bases, buffers), stabilizers (e.g., ascorbic acid) or isotonizing agents (e.g., sodium chloride).

The term “pharmaceutically acceptable salt” as used herein refers to those carboxylate salts or acid addition salts of the compounds of the present invention which are, within the scope of sound medical judgement, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylaamine, dimethylamine, trimethylamine, triethylamine, ethylamine, and the like. (See, for example, Berge S. M., et al., Pharmaceutical Salts, J. Pharm. Sci. 66:1-19 (1977) which is incorporated herein by reference.)

In the first step of the present method of imaging, a labeled compound of Formula IV, wherein L is selected from the group consisting of L1, L1′, L2, L2′, L3, L3′, L4, L5, L6, L6′, L7, L7′, L8 and L9, is introduced into a tissue or a patient in a detectable quantity. The compound is typically part of a pharmaceutical composition and is administered to the tissue or the patient by methods well known to those skilled in the art.

For example, the compound can be administered either orally, rectally, parenterally (intravenous, by intramuscularly or subcutaneously), intracisternally, intravaginally, intraperitoneally, intravesically, locally (powders, ointments or drops), or as a buccal or nasal spray.

The administration of the labeled compound to a patient can be by a general or local administration route. For example, the labeled compound may be administered to the patient such that it is delivered throughout the body. Alternatively, the labeled compound can be administered to a specific organ or tissue of interest. For example, it is desirable to locate and sites and receptors of interest to diagnose or track the progress of a disease in a patient.

The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The term “patient” means humans and other animals. Those skilled in the art are also familiar with determining the amount of time sufficient for a compound to become associated with amyloid deposits. The amount of time necessary can easily be determined by introducing a detectable amount of a labeled compound of Formulae IV into a patient and then detecting the labeled compound at various times after administration.

The term “associated” means a chemical interaction between the labeled compound and the site or receptor of interest. Examples of associations include covalent bonds, ionic bonds, hydrophilic-hydrophilic interactions, hydrophobic-hydrophobic interactions, and complexes.

Those skilled in the art are familiar with the various ways to detect labeled compounds. For example, magnetic resonance imaging (MRI), positron emission tomography (PET), or single photon emission computed tomography (SPECT) can be used to detect radiolabeled compounds. The label that is introduced into the compound will depend on the detection method desired. For example, if PET is selected as a detection method, the compound must possess a positron-emitting atom, such as ¹⁸F.

The radioactive diagnostic agent should have sufficient radioactivity and radioactivity concentration which can assure reliable diagnosis. For instance, in case of the radioactive metal being technetium-99m, it may be included usually in an amount of 0.1 to 50 mCi in about 0.5 to 5.0 ml at the time of administration. The amount of a compound of Formulae IV, wherein L is selected from the group consisting of L1, L1′, L2, L2′, L3, L3′, L4, L5, L6, L6′, L7, L7′, L8 and L9 may be such as sufficient to form a stable chelate compound with the radioactive metal.

The thus formed chelate compound as a radioactive diagnostic agent is sufficiently stable, and therefore it may be immediately administered as such or stored until its use. When desired, the radioactive diagnostic agent may contain any additive such as pH controlling agents (e.g., acids, bases, buffers), stabilizers (e.g., ascorbic acid) or isotonizing agents (e.g., sodium chloride).

### EXAMPLES

All reagents used in the synthesis were commercial products used without further purification unless otherwise indicated. ¹H NMR spectra were obtained on a Bruker DPX spectrometer (200 MHz) in CDCl₃ unless otherwise indicated. Chemical shifts are reported as δ values (parts per million) relative to internal TMS. Coupling constants are reported in hertz. The multiplicity is defined by s (singlet), d (doublet), t (triplet), br (broad), m (multiplet). High resolution electron ionization (HREI) mass spectra were performed at the McMaster Regional Centre for Mass Spectrometry using a Micromass/Waters GCT instrument (GC-EI/CI Time of Flight Mass Spectrometer).

### Example 1

### Synthesis of 2-phenylbenzothiozole (PE3) derivatives

Compound 4 (2-phenylbenzothiozole (PIB) core) was prepared using Mathis and co-workers approach (26). Monomethylation was accomplished via standard reported procedures (27) to yield 4 that was used in subsequent steps.

1. General Procedure for the O-alkylation of 4

To a solution of 4 (1 eq) in anhyd. N′,N″-dimethylformamide (2 mL/0.1 mmol of 4) in a microwavable vial (from Biotage) was added anhyd. cesium carbonate (2.5 eq) and the mixture stirred at room temperature under argon for 30 min. Alkylating agent (1.2 eq) followed by sodium iodide (1.5 eq) were then added, the vial was sealed and subjected to microwave irradiation (Biotage Initiator system). The microwave conditions were, 200° C. for 10 min. with 10 sec. pre-stirring and with fixed hold time “on”. After cooling the reaction mixture to room temperature the vial was opened, the contents were transferred to a round-bottom flask and the volatiles were removed under reduced pressure. The residue was extracted with ethyl acetate (3×10 mL) and the ethyl acetate layer was washed with water (1×10 mL) and brine (1×10 mL). The organic layer, after drying over anhyd. magnesium sulfate, was evaporated and the residue was purified by preparative thin layer chromatography on silica to afford the corresponding PEGylated derivative.

2. Preparation of Compounds 5(a-d)

Treatment of 4 with the fluoromesylates according to the general procedure afforded compounds 5(a-d).

2-[4′-(methylamino)phenyl]-6-[2-(2-fluoroethoxy)-ethoxy]benzothiazole (5a) (PTLC, 50% ethyl acetate in hexane, 84%). ¹H NMR (200 MHz, CDCl₃): δ 7.83-7.89 (3H, m), 7.33 (1H, d, J=2.5 Hz), 7.06 (1H, dd, J=8.9, 2.5 Hz), 6.63 (2H, d, J=8.9 Hz), 4.60 (2H, dt, J=47.6, 4.2 Hz), 4.21 (2H, t, J=4.5 Hz), 3.89-3.94 (3H, m), 3.76 (1H, d, J=4.2 Hz), 2.90 (3H, s). HRMS (ED) m/z calcd. for [C₁₈H₁₉FN₂O₂S]⁺ 346.1151, found 346.1141.

2-[4′-(methylamino)phenyl]-6-{2-[2-(2-fluoroethoxy)-ethoxy]ethoxy}benzothiazole (5b) (PTLC, 60% ethyl acetate in hexane, 78%). ¹H NMR (200 MHz, CDCl₃): δ 7.82-7.88 (3H, m), 7.32 (1H, d, J=2.5 Hz), 7.05 (1H, dd, J=8.8, 2.5 Hz) 6.63 (2H, d, J=8.8 Hz), 4.56 (2H, dt, J=47.6, 4.2 Hz), 4.19 (2H, t, J=4.5 Hz), 3.65-3.88(8H, m), 2.89 (3H, s). HRMS (EI) m/z calcd. for [C₂₀H₂₃FN₂O₃S]⁺ 390.1413, found 390.1386

2-[4′-(methylamino)phenyl]-6-{2-[2-(2-{2-[2-(2-fluoroethoxy)-ethoxy]ethoxy}ethoxy)ethoxy]ethoxy}benzothiazole (5c) (PTLC: 80% ethyl acetate in hexane, Yield 72%). ¹H NMR (200 MHz, CDCl₃): δ 7.82-7.87 (3H, m), 7.33 (1H, d, J=2.4 Hz), 7.05 (1H, dd, J=8.8, 2.4 Hz), 6.63 (2H, d, J=8.8 Hz), 4.54 (2H, dt, J=47.6, 4.1 Hz), 4.18 (2H, t, J=4.5 Hz), 3.65-3.90 (20H, m), 2.89 (3H, s). HRMS (EI) m/z calcd for [C₂₆H₃₅FN₂O₆S]⁺ 522.2200, found 522.2175.

2-[4′-(methylamino)phenyl]-6-[2-(2-{2-[2-(2-{2-[2-(2-fluoroethoxy)-ethoxy]ethoxy}ethoxy)ethoxy]ethoxy}ethoxy)ethoxy]benzothiazole (5d) (PTLC: ethyl acetate, Yield 71%). ¹H NMR (200 MHz, CDCl₃): δ 7.81-7.87 (3H, m), 7.33 (1H, d, J=2.4 Hz), 7.05 (1H, dd, J=8.8, 2.4 Hz), 6.63 (2H, d, J=8.8 Hz), 4.55 (2H, dt, J=47.7, 4.2 Hz), 4.19 (2H, t, J=4.4 Hz), 3.63-3.90 (28H, m), 2.89 (3H, s). HRMS (EI) m/z calcd for [C₃₀H₄₃FN₂O₈S]⁺ 610.2724, found 610.2705.

3. Preparation of Compounds 6(a-c)

Treatment of 4 with hydroxymesylates according to the general procedure afforded compounds 6(a-c).

2-{2-[2-(4-methylamino-phenyl)-benzothiazol-6-yloxy]-ethoxy}-ethanol (6a) (PTLC, 1% methanol in dichloromethane, 82%). ¹H NMR (200 MHz, CDCl₃): δ 7.85-7.89 (3H, m), 7.33 (1H, d, J=2.4 Hz), 7.06 (1H, dd, J=8.8, 2.4 Hz), 6.64 (2H, d, J=8.8 Hz), 4.20 (2H, d, J=4.3 Hz), 3.90 (2H, d, J=4.6 Hz), 3.69-3.78 (m, 4H), 2.90 (s, 3H). HRMS (EI) m/z calcd for [C₁₈H₂₀N₂O₃S]⁺ 344.1195, found 344.1188

2-(2-{2-[2-(4-methylamino-phenyl)-benzothiazol-6-yloxyl-ethoxy}-ethoxy)-ethanol (6b) (PTLC, 1% methanol in dichloromethane, 74%). ¹H NMR (200 MHz, CDCl₃): δ 7.83-7.88 (3H, m), 7.31 (1H, d, J=2.5 Hz), 7.05 (1H, dd, J=8.8, 2.5 Hz) 6.63 (2H, d, J=8.8 Hz), 4.19 (2H, t, J=4.5 Hz), 3.88 (2H, t, J=4.6 Hz) 3.58-3.78(8H, m), 2.90 (3H, s) HRMS (EI) m/z calcd for [C₂₀H₂₄N₂O₄S]⁺ 388.1457, found 388.1444.

2-(2-{2-[2-(2-{2-[2-(4-methylamino-phenyl)-benzothiazol-6-yloxy]-ethoxy}-ethoxy)-ethoxyl-ethoxy}-ethoxy)-ethanol (6c) (PTLC, 2% methanol in dichloromethane, 66%). ¹H NMR (200 MHz, CDCl₃): δ 7.83-7.88 (s, 3H), 7.33 (1H, d, J=2.41 Hz), 7.06 (1H, dd, J=8.8, 2.5 Hz), 6.63 (2H, d=8.8 Hz), 4.19 (2H, t, J=4.5 Hz), 3.88 (2H, t, J=4.8 Hz), 3.56-3.53 (20H, m), 2.90 (3H, s). HRMS (EI) m/z calcd for [C₂₆H₃₆N₂O₇S]⁺ 520.2243, found 520.2282.

4. Preparation of Compounds 7(a-c)

Alkylation of 4 with tert-butyldimethylsilyl protected mesylates as per the general procedure afforded compounds 7(a-c).

2-[4′-(methylamino)phenyl]-6-[2-(2-tert-butyldimethylsilyloxy-ethoxy)-ethoxy]benzothiazole (7a) (PTLC, 50% ethyl acetate in hexane, 70%). ¹H NMR (200 MHz, CDCl₃): δ 7.84-7.88 (3H, m), 7.32 (1H, d, J=2.4 Hz), 7.06 (1H, dd, J=8.8, 2.4 Hz), 6.64 (2H, d, J=8.8 Hz), 4.20 (2H, d, J=4.3 Hz), 3.90 (2H, d, J=4.6 Hz), 3.64-3.78 (m, 4H), 2.90 (s, 3H), 0.88 (9H, s), 0.05 (6H, s).

2-[4′-(methylamino)phenyl]-6-{2-[2-(2-tert-butyldimethylsilyloxy-ethoxy)-ethoxy]ethoxy}benzothiazole (7b) (PTLC, 60% ethyl acetate in hexane, 62 %). ¹H NMR (200 MHz, CDCl₃): δ 7.82-7.87 (3H, m), 7.30 (1H, d, J=2.5 Hz), 7.05 (1H, dd, J=8.8, 2.5 Hz) 6.62 (2H, d, J=8.8 Hz), 4.21 (2H, t, J=4.5 Hz), 3.88 (2H, t, J=4.6 Hz) 3.58-3.74(8H, m), 2.90 (3H, s), 0.88 (9H, s), 0.05 (6H, s).

2-[4′-(methylamino)phenyl]-6-{2-[2-(2-{2-[2-(2-tert-butyldimethylsilyloxy-ethoxy)-ethoxy]ethoxy}ethoxy)ethoxylethoxy}benzothiazole (7c) (PTLC, 80% ethyl acetate in hexane, 55%). ¹H NMR (200 MHz, CDCl₃): δ 7.85-7.89 (3H, m), 7.32 (1H, d, J=2.4 Hz), 7.05 (1H, dd, J=8.4, 2.4 Hz), 6.64 (2H, d, J=8.4 Hz), 4.19 (2H, t, J=4.7 Hz), 3.88 (2H, t, J=4.9 Hz), 3.44-3.77 (20H, m), 2.90 (3H, s), 0.88 (9H, s), 0.05 (6H, s).

5. General Procedure for the Preparation of Compounds 10(a-c)

General procedure for Boc protection to from 7′(a-c): Compound 7(a-c) (1 eq.) was dissolved in anhydrous tetrahydrofuran (10 mL/mmol of 7) and to the resulting solution ditert-butyldicarbonate (2 eq) and 4-dimethylaminopyridine (catalytic) were added and the mixture heated to reflux. After 16 h another batch of ditert-butyldicarbonate (1 eq) was added and the mixture was further refluxed for another 20 h. The reaction mixture was then cooled to rt and the solvent was removed under reduced pressure. The residue was taken in ethyl acetate (25 mL/mmol of 6) washed successively with water (1×10 mL) and brine(1×10 mL) and dried over anhyd. magnesium sulfate. The residue after removing the solvent was purified by PTLC.

2-[4′-(N-tert-butyloxycabonyl-N-methylamino)phenyl]-6-[2-(2-tert-butyldimethylsilyloxy-ethoxy)-ethoxy]benzothiazole (7′a) (PTLC, 20% ethyl acetate in hexane, 55%). ¹H NMR (200 MHz, CDCl₃): δ 7.91-8.01 (3H, m), 7.34-7.38 (3H, m), 7.08 (1H, dd, J=8.8, 2.4 Hz), 4.22 (2H, d, J=4.3 Hz), 3.89 (2H, d, J=4.6 Hz), 3.60-3.76 (m, 4H), 3.02 (s, 3H), 1.47 (9H, s), 0.88 (9H, s), 0.05( 6H, s).

2-[4′-(N-tert-butyloxycarbonyl-N-methylamino)phenyl]-6-{2-[2-(2-tert-butyldimethylsilyloxy-ethoxy)-ethoxy]ethoxy}benzothiazole (7′b) (PTLC, 30% ethyl acetate in hexane, 48%). ¹H NMR (200 MHz, CDCl₃): δ 7.90-7.99 (3H, m), 7.34-7.37 (3H, m), 7.06 (1H, dd, J=8.6, 2.5 Hz) 4.20 (2H, t, J=4.5 Hz), 3.88 (2H, t, J=4.6 Hz) 3.54-3.69 (8H, m), 3.01 (3H, s), 1.46 (9H, s), 0.88 (9H, s), 0.05 (6H, s).

2-[4′-(N-tert-butyloxycarbonyl-N-methylamino)phenyl]-6-{2-[2-(2-{2- [2-(2-tert-butyldimethylsilyloxy-ethoxy)-ethoxy]ethoxy]ethoxy)ethoxy]ethoxy}benzothiazole (7′c) (PTLC, 50% ethyl acetate in hexane, 40%). ¹H NMR (200 MHz, CDCl₃): δ 7.92-8.01 (3H, m), 7.36-7.40 (3H, m), 7.05 (1H, dd, J=8.4, 2.4 Hz), 4.20 (2H, t, J=4.7 Hz), 3.88 (2H, t, J=4.9 Hz), 3.44-3.77 (20H, m), 3.02 (3H, s), 1.47 (9H, s), 0.88 (9H, s), 0.05 (6H, s).

General procedure for deprotection followed by preparation of the mesylate derivatives 10(a-c): tert-Butyl carbonate (BOC) protected compound 7′(a-c) was dissolved in anhyd. tetrahydrofuran (3 mL/0.1 mmol of 7′) and the resulting solution was cooled to 0° C. Tetrabutylammonium fluoride (2 eq, 1M in tetrahydrofuran) was added to the ice cold solution and stirred at that temperature for 15 min. and then at room temperature for 2 h. Pulled the solvent off and the residue was extracted in ethyl acetate (3×10 mL). The ethyl acetate layer was washed with water (1×10 mL), brine (1×10 mL) and dried over anhyd. magnesium sulfate. The residue after removal of the solvent was used as such for the subsequent step without purification.

The crude from the above step was dissolved in anhyd. dichloromethane (1 mL/0.1 mmol of 7′) along with anhyd. triethylamine (4 eq) and the mixture was cooled in an ice-acetone bath (˜−5° C.). Methanesulfonyl choride (3 eq) was then added and the mixture was stirred at that temperature for 15 min. Reaction mixture was brought to room temperature gradually and stirred for an additional 2 hours. It was then quenched with ice and extacted with dichloromethane (3×5 mL). The organic layer after drying over anhyd. magnesium sulfate was purified by PTLC.

2-[4′-(N-tert-butyloxycabonyl-N-methylamino)phenyl]-6-[2-(2-methylsulfonyloxy-ethoxy)-ethoxy]benzothiazole (10a) (PTLC, 1% methanol in dichlormethane, 92%). ¹H NMR (200 MHz, CDCl₃): δ 7.91-8.01 (3H, m), 7.34-7.38 (3H, m), 7.08 (1H, dd, J=8.8, 2.4 Hz), 4.39-4.44 (2H, m), 4.19-4.23 (2H, m), 3.84-3.89 (4H, m), 3.30 (3H, s), 3.05 (3H, s), 1.47 (9H, s).

2-[4′-(N-tert-butyloxycarbonyl-N-methylamino)phenyl]-6-{2-[2-(2-methylsulfonyloxy-ethoxy)-ethoxy]ethoxy}benzothiazole (10b) (PTLC, 1% methanol in dichloromethane, 95%). ¹H NMR (200 MHz, CDCl₃): δ 7.90-8.01 (3H, m), 7.33-7.38 (3H, m), 7.09 (1H, dd, J=8.8, 2.5 Hz), 4.35-4.39 (2H, m), 4.17-4.22 (2H, m), 3.86-3.91 (2H, m), 3.69-3.80 (6H, m), 3.31 (3H, s), 3.05 (3H, s), 1.47 (9H, s)

2-[4′-(N-tert-butyloxycarbonyl-N-methylamino)phenyl]-6-{2-[2-(2-12-[2-(2-methylsulfonyloxy-ethoxy)-ethoxy]ethoxy}ethoxy)ethoxy]ethoxy}benzothiazole (10c) (PTLC, 2% methanol in dichloromethane, 90%). ¹H NMR (200 MHz, CDCl₃): δ 7.91-8.01 (3H, m), 7.34-7.38 (3H, m), 7.11 (1H, dd, J=8.8, 2.4 Hz), 4.33-4.38 (2H, m), 4.18-4.23 (2H, m), 3.87-3.92 (2H, m), 3.62-3.78 (18 H, m), 3.31 (3H, s), 3.06 (3H, s), 1.47 (9H, s).

### Example 2

### 1. Preparation of [2-(4-dimethylaminophenyl)-vinyl]-benzoxazol derivatives

2-(2-(4-dimethylaminophenyl)vinyl)-benzooxazol-6-ol (3′): 2-methyl-benzoxazol-6-ol (prepared following Schreiner and coworkers method (28)) (1.7 mmol) was dissolved in anhydrous tetrahydrofuran (8 mL) and cooled to 0° C. Trimethylsilyl chloride (1.8 mmol) and diisopropylethylamine (1.84 mmol) were then added and the resultant solution stirred for 2 hours at room temperature. After cooling to −78° C., sodium hexamethyldisilazane (11.7 mmol, 1.0 M solution in tetrahydrofuran) was added slowly over 1.5 hours and then stirred at −78° C. for an additional hour. 4-(dimethylamino)-benzaldehyde was then added and the reaction allowed to warm to room temperature overnight. The reaction was then poured into a 1M solution of sodium hydrogen sulfate and extracted with ethyl acetate. The organic layers were then washed with brine, dried over magnesium sulfate and concentrated to yield a yellow solid that was purified using column chromatography (3% methanol in dichloromethane). Yield: 45%. ¹H NMR (200 MHz, CDCl₃): δ 7.57 (2H, d, J=8.9 Hz), 7.55 (1H, d, J=16.0 Hz), 7.43 (1H, d, J=8.5 Hz), 6.99 (1H, d, J=2.1 Hz), 6.89 (1H, d, J=16.0 Hz), 6.78 (1H, dd, J=8.5, 2.1 Hz), 6.73 (2H, d, J=8.9 Hz), 2.98 (6H, s). HRMS (EI) m/z calcd. for [C₁₇H₁₆N₂O₂]⁺ 280.1212, found 280.1205.

2. General Procedure for O-alkylation of 3′.

To a solution of (3′) (1 eq) in anhydrous N′,N″-dimethylformamide (2 mL) in a microwavable vial (from Biotage) was added potassium carbonate (3.0 eq) and alkylating agent (1.2-1.5 eq). The vial was sealed and subjected to microwave irradiation (Biotage Initiator system) at 200° C. for 10 min. with 10 sec. pre-stirring and with fixed hold time “on”. After cooling the reaction mixture to room temperature the vial was opened, the contents poured into water and extracted with ethyl acetate (3×10 mL). The ethyl acetate layer was washed with water (2×10 mL) and brine (2×10 mL). The organic phase was then dried over anhyd. sodium sulfate, and evaporated. The residue was purified by preparative thin layer chromatography on silica to afford the corresponding PEGylated derivative (8a-d).

6-(2-fluoroethoxy)-[2-(4-dimethylaminophenyl)-vinyl]-benzooxazol (8a): Yield: 68%. ¹H NMR (200 MHz, CDCl₃): δ 7.64 (1H, d, J=16.2 Hz), 7.54 (1H, d, J=8.7 Hz), 7.47 (2H, d, J=8.8 Hz), 7.06 (1H, d, J=2.3 Hz), 6.93 (1H, dd, J=8.7, 2.3 Hz), 6.80 (1H, d, J=16.2 Hz), 6.72 (2H, d, J=8.8 Hz), 4.78 (2H, dt, J=47.4, 4.0 Hz), 4.26 (2H, dt, J=27.7, 4.0 Hz), 3.02 (6H, s). HRMS (EI) m/z calcd. for [C₁₉H₁₉FN₂O₂]⁺ 326.1434, found 326.1431.

6-(2-(2-(2-fluoroethoxy)-ethoxy)-ethoxy)-[2-(4-dimethylaminophenyl)-vinyl]-benzooxazol (8b): Yield: 71%. ¹H NMR (200 MHz, CDCl₃): δ 7.63 (1H, d, J=16.2 Hz), 7.52 (1H, d, J=8.8 Hz), 7.47 (2H, d, J=9.0 Hz), 7.06 (1H, d, J=2.1 Hz), 6.92 (1H, dd, J=8.8, 2.1 Hz), 6.80 (1H, d, J=16.2 Hz), 6.72 (2H, d, J=9.0 Hz), 4.57 (2H, dt, J=47.6, 4.1 Hz), 4.19 (2H, t, J=4.5 Hz), 3.92-3.67 (10H, m), 3.02 (6H, s). HRMS (EI) m/z calcd. for [C₂₃H₂₇FN₂O₄]⁺ 414.1955, found 414.1946.

6-(2-(2-(2-(2-(2-(2-fluoroethoxy)-ethoxy)-ethoxy)-ethoxy)-ethoxy)-ethoxy)-[2-(4-dimethylaminophenyl)-vinyl]-benzooxazol (8c): Yield: 66%. ¹H NMR (200 MHz, CDCl₃): δ 7.63 (1H, d, J=16.2 Hz), 7.51 (1H, d, J=8.1 Hz), 7.46 (2H, d, J=8.7 Hz), 7.05 (1H, d, J=2.1 Hz), 6.91 (1H, dd, J=8.1, 2.1 Hz), 6.80 (1H, d, J=16.2 Hz), 6.71 (2H, d, J=8.7 Hz), 4.54 (2H, dt, J=47.5, 3.8 Hz), 4.17 (2H, t, J=5.1 Hz), 3.90-3.65 (20H, m), 3.02 (6H, s). HRMS (EI) m/z calcd. for [C₂₉H₃₉FN₂O₇]⁺ 546.2741, found 546.2740.

6-(2-(2-(2-(2-(2-(2-(2-(2-fluoroethoxy)-ethoxy)-ethoxy)-ethoxy)-ethoxy)-ethoxy)-ethoxy)-ethoxy)-[2-(4-dimethylaminophenyl)-vinyl]-benzooxazol (8d): Yield: 95%. ¹H NMR (200 MHz, CDCl₃): δ 7.62 (1H, d, J=16.2 Hz), 7.50 (1H, d, J=8.6 Hz), 7.48 (2H, d, J=8.9 Hz), 7.08 (1H, d, J=2.2 Hz), 6.92 (1H, dd, J=8.6, 2.2 Hz), 6.80 (1H, d, J=16.2 Hz), 6.73 (2H, d, J=8.9 Hz), 4.53 (2H, dt, J=47.7, 4.0 Hz), 4.17 (2H, t, J=4.39 Hz), 3.87-3.59 (30H, m), 3.02 (6H, s). HRMS (EI) m/z calcd. for [C₃₃H₄₇FN₂O₉]⁺ 634.3266, found 634.3242.

3. Preparation of Hydroxy Derivative (9)

To a solution of (3′) (1 eq) in anhydrous N′,N″-dimethylformamide (2 mL) in a microwavable vial (from Biotage) was added potassium carbonate (3.0 eq) and 2-(2-(2-chloroethoxy)ethoxy)ethanol (1.5 eq). The vial was sealed and subjected to microwave irradiation (Biotage Initiator system) at 200° C. for 10 min. with 10 sec. pre-stirring and with fixed hold time “on”. After cooling the reaction mixture to room temperature the vial was opened, the contents poured into water and extracted with ethyl acetate (3×10 mL). The ethyl acetate layer was washed with water (2×10 mL) and brine (2×10 mL). The organic phase was then dried over anhyd. sodium sulfate, and evaporated. The residue was purified by silica prep TLC (25% hexanes in ethyl acetate) to afford the corresponding hydroxy PEGylated derivative (9) in 80% yield. ¹H NMR (200 MHz, CDCl₃): δ 7.61 (1H, d, J=16.2 Hz), 7.52 (1H, d, J=8.8 Hz), 7.48 (2H, d, J=9.0 Hz), 7.05 (1H, d, J=2.2 Hz), 6.92 (1H, dd, J=8.8, 2.2 Hz), 6.80 (1H, d, J=16.2 Hz), 6.72 (2H, d, J=9.0 Hz), 4.17 (2H, t, J=4.4 Hz), 3.88 (2H, t, J=4.4 Hz), 3.76-3.59 (8H, m), 3.00 (6H, s).

4. Preparation of Mesylate Labeling Precursor (11)

Compound 9 was dissolved in dichloromethane followed by the addition of triethylamine (4.0 eq). Methanesulfonyl chloride was then added via a syringe and the resultant solution stirred for 3 hours at room temperature. The solution was then poured into water and extracted with dichloromethane, washed with brine and dried over sodium sulfate. The residue was purified via silica gel PTLC (25% hexanes in ethyl acetate) to afford the mesylated precursor (11) in 75% yield. ¹H NMR (200 MHz, CDCl₃): δ 7.63 (1H, d, J=16.2 Hz), 7.52 (1H, d, J=8.8 Hz), 7.48 (2H, d, J=9.0 Hz), 7.05 (1H, d, J=2.1 Hz), 6.92 (1H, dd, J=8.8, 2.1 Hz), 6.80 (1H, d, J=16.2 Hz), 6.72 (2H, d, J=9.0 Hz), 4.37 (2H, t, J=4.4 Hz), 4.19 (2H, t, J=4.4 Hz), 3.87 (2H, t, J=4.3 Hz), 3.79-3.61 (6H, m), 3.05 (3H, s), 3.02 (6H, s).

### Example 3

### 1. Synthesis of 6-iodo-2-(4′-dimethylamino)phenyl-imidazo[1,2-a]pyridine (IMPY) (2) derivatives

Preparation of 2 (IMPY core) has been described elsewhere (29). The general procedure for the synthesis of 6-FPEG substituted-SPY conjugates was accomplished using the following procedure:

Conventional synthesis: The mixture of 2 (prepared as reported previously reported (29)), fluoro-polyglycols (2-5 eq.), CuI (10% mol), Cs₂CO₃ (2 eq.), 1,10-phenanthroline (20% mol) in Toluene (1 mL/0.1 mmol 2) was stirred in a sealed tube for 48 h. Solvent was removed and PTLC [Ethyl Acetate or dichloromethane-methanol (95:5) as developing solvent] gave the desired product (Yield: 17-60% depending on the glycol used).

Microwave synthesis: The mixture of reactants and reagents described above in a sealed tube was put in the microwave oven—condition: 170° C., 60 min, normal absorption level. (Yields were similar to those used the conventional synthesis).

6-(2-fluoroethoxy)-2-(4-dimethylamino-)phenyl-imidazo [1,2-a]pyridine (12a): Yield: 17%. ¹H NMR (200 MHz, CDCl₃): δ 7.78 (2H, d, J=8.8 Hz), 7.68 (1H, d, J=2.2 Hz), 7.67 (1H, s), 7.50 (1H, d, J=9.7 Hz), 6.96 (1H, dd, J=9.7, 2.2 Hz), 6.74 (2H, d, J=8.8 Hz), 4.75 (2H, dt, J=47.7, 4.1 Hz), 4.16 (2H, dt, J=25.9, 4.1 Hz), δ 2.99 (6H, s). HRMS (EI) m/z calcd. for [C₁₇H₁₉FN₃O]⁺ (M+H)⁺ 300.1512, found 300.1500.

6-(2-fluoroethoxy-ethoxy)-2-(4-dimethylamino-)phenyl-imidazo[1,2-a]pyridine (12b): Yield: 59%. ¹H NMR (200 MHz, CDCl₃): δ 7.78 (2H, d, J=8.8 Hz). 7.66 (1H, d, J=2.2 Hz), 7.64 (1H, s), 7.46 (1H, d, J=9.7 Hz), 6.94 (1H, dd, J=9.7, 2.2 Hz), 6.76 (2H, d, J=8.8 Hz), 4.59 (2H, dt, J=47.6, 4.1 Hz), 4.08 (2H, t, J=4.2 Hz), 3.88 (2H, t, J=4.2 Hz), 3.80 (2H, dt, J=25.9, 4.1 Hz), 2.98 (6H, s). HRMS (EI) m/z calcd. for [C₁₉H₂₃FN₃O₂]⁺ (M+H)⁺ 344.1774, found 344.1768.

6-(2-fluoroethoxy-ethoxy-ethoxy)-2-(4-dimethylamino-)phenyl-imidazo[1,2-a]pyridine (12c): Yield: 60%. ¹H NMR (200 MHz, CDCL₃): δ 7.77 (2H, d, J=8.8 Hz), 7.65 (1H, d, J=2.2 Hz), 7.63 (1H, s), 7.45 (1H, d, J=9.7 Hz), 6.93 (1H, dd, J=9.7, 2.2 Hz), 6.75 (2H, d, J=8.8 Hz), 4.54 (2H, dt, J=47.7, 4.1 Hz), 4.06 (2H, t, J=4.6 Hz), 3.82 (2H, t, J=4.6 Hz), 3.70-3.59 (6H, m), 2.97 (6H, s). HRMS (EI) m/z calcd. for [C₂₁H₂₇FN₃O₃]⁺ (M+H)⁺ 388.2036, found 388.2032.

6-(2-fluoroethoxy-ethoxy-ethoxy-ethoxy-ethoxy-ethoxy)-2-(4-dimethylamino-)phenyl-imidazo[1,2-a]pyridine (12d): Yield: 18%. ¹H NMR (200 MHz, CDCL₃): δ 7.78 (2H, d, J=8.8 Hz), 7.71 (1H, d, J=1.9 Hz), 7.67 (1H, s), 7.48 (1H, d, J=9.7 Hz), 6.93 (1H, dd, J=9.7, 2.2 Hz), 6.76 (2H, d, J=8.8 Hz), 4.53 (2H, dt, J=47.7, 4.1 Hz), 4.09 (2H, t, J=4.6 Hz), 3.85 (2H, t, J=4.6 Hz), 3.89-3.64 (18H, m), 2.98 (6H, s). HRMS (EI) m/z calcd. for [C₂₇H₃₉FN₃O₆] (M+H)⁺ 520.2823, found 520.2808.

6-(2-fluoroethoxy-ethoxy-ethoxy-ethoxy-ethoxy-ethoxy-ethoxy-ethoxy)-2-(4-dimethylamino-) phenyl-imidazo[1,2-a]pyridine (12e): Yield: 58%. ¹H NMR (200 MHz, CDCL₃): δ 7.75 (2H, d, J=8.8 Hz), 7.68 (1H, d, J=1.9 Hz), 7.62 (1H, s), 7.52 (1H, d, J=9.7 Hz), 6.95 (1H, dd, J=9.7, 2.2 Hz), 6.71 (2H, d, J=8.8 Hz), 4.50 (2H, dt, J=47.7, 4.0 Hz), 4.07 (2H, t, J=4.6 Hz), 3.64-3.85 (28H, m), 2.95 (6H, s). HRMS (EI) m/z calcd. for [C₃₁H₄₇FN₃O₈]⁺ (M+H)⁺ 608.3347, found 608.3329.

### Example 4

### Radiochemistry

1. General Procedure for ¹⁸F Labeling of 10(a) Using Oil Bath Heating:

[¹⁸F]Fluoride was produced by a cyclotron using ¹⁸O(p,n)¹⁸F reaction. An [¹⁸O]-enriched aqueous solution of [¹⁸F]Fluoride was passed through a Sep-Pak Light quaternary methyl ammonium (QMA) cartridge and the cartridge dried by airflow. The ¹⁸F activity was then eluted using 1.2 mL of a Kryptofix 222/potassium carbonate solution, which is made up of 22 mg of Kryptofix 222 and 4.6 mg of potassium carbonate in acetonitrile/water 1.77/0.23. The solvent was removed under a stream of nitrogen at 120° C. and the residue azeotropically dried twice with 1 mL of anhydrous acetonitrile also at 120° C. Mesylate precursor (10a) (0.5, 1, 3, and 6 mg) was then dissolved in 0.2 mL of dimethyl sulfoxide and added to the reaction vessel containing the dry ¹⁸F. The reaction was then heated at 75, 90, 105 or 120° C. for 4, 8, 12 or 16 minutes. Water (2 mL) was then added and the resultant solution loaded onto an Oasis HLB cartridge previously washed with 2×3 mL ethanol and 2×3 mL of water. The cartridge was subsequently washed with 4 mL of water and the crude product eluted with 2 mL of acetonitrile, which was then injected onto the HPLC for purification using a Phenomenex Gemini C18 semi-prep column [(5.0×250 mm, 5 μm); Acetonitrile/water 70/30; flow rate 3 mL/min](Analytical HPLC conditions: Phenomenex Gemini C18 Analytical column [(5.0×250 mm, 5 μm); Acetonitrile/water 80/20; flow rate 1 mL/min). The retention time of the major hydrolysis by product (Rt=3.6 min) was well resolved from the ¹⁸F labeled product (Rt=5.2 min), which was isolated in >99% radiochemical purity.

2. General Procedure for ¹⁸F Labeling of 10b, 10c, and 11

Compound 10b, 10c and 11 were labeled with ¹⁸F using the above described procedure, with heating for 4 minutes at 120° C. The crude reaction was HPLC purified using a Phenomenex Gemini C18 semi-prep column [(5.0×250 mm, 5 μm); Acetonitrile/water 70/30; flow rate 3 mL/min]

¹⁸F]5b from precursor 10b: Retention time of ¹⁸F-labeled product was 8.0 min., well separated from the major hydrolysis by-product (Rt=4.2 min.). The product was isolated in 35% radiochemical yield (decay corrected) and greater than 98% radiochemical purity.

¹⁸F]5c from precursor 10c: Retention time of ¹⁸F-labeled product was 8.1 min., well separated from the major hydrolysis by-product (Rt=4.5 min.). The product was isolated in 11% radiochemical yield (decay corrected) and greater than 98% radiochemical purity.

[¹⁸F]8b from precursor 11: (HPLC conditions: Phenomenex Gemini C18 semi-prep column [(5.0×250 mm, 5 μm); Acetonitrile/water 60/40; flow rate 3 mL/min)

Retention time of ¹⁸F-labeled product was 28.0 min., well separated from the major hydrolysis by-product (Rt=11.8 min.). The product was isolated in 23% radiochemical yield (decay corrected) and greater than 98% radiochemical purity. Purified [¹⁸F]8b was injected periodically after purification. Formation of a second peak increased to 50% of the parent compound.

### Example 5

### Binding Studies

Postmortem brain tissues were obtained from AD patients at autopsy, and neuropathological diagnosis was confirmed by current criteria (NIA-Reagan Institute Consensus Group, 1997). Homogenates were then prepared from dissected gray matters from AD patients in phosphate buffered saline (PBS, pH 7.4) at the concentration of approximately 100 mg wet tissue/ml (motor-driven glass homogenizer with setting of 6 for 30 sec). The homogenates were aliquoted into 1 ml-portions and stored at −70° C. for 6-12 months without loss of binding signal.

As reported previously, [¹²⁵I]IMPY (13), with 2,200 Ci/mmol specific activity and greater than 95% radiochemical purity, was prepared using the standard iododestannylation reaction and purified by a simplified C-4 mini column (13). Binding assays were carried out in 12×75 mm borosilicate glass tubes. The reaction mixture contained 50 μl of brain homogenates (20-50 μg), 50 μl of [¹²⁵I]IMPY (0.04-0.06 nM diluted in PBS) and 50 μl of inhibitors (10⁻⁵-10⁻¹⁰ M diluted serially in PBS containing 0.1% bovine serum albumin, BSA) in a final volume of 1 ml. Nonspecific binding was defined in the presence of IMPY (600 nM) in the same assay tubes. The mixture was incubated at 37° C. for 2 hr and the bound and the free radioactivity were separated by vacuum filtration through Whatman GF/B filters using a Brandel M-24R cell harvester followed by 2×3 ml washes of PBS at room temperature. Filters containing the bound ¹²⁵I ligand were assayed for radioactivity content in a gamma counter (Packard 5000) with 70% counting efficiency. Under the assay conditions, the specifically bound fraction was less than 15% of the total radioactivity. The results of inhibition experiments were subjected to nonlinear regression analysis using EBDA by which Kᵢ values were calculated and are shown in Table 1.

TABLE 1
Inhibition constants (Ki, nM) on binding of [¹²⁵I]IMPY
to Ab aggregates of AD brain homogenates*

| Compounds | Kᵢ (nM) | logP⁺ | Compounds | Kᵢ (nM) | logP⁺ |
|---|---|---|---|---|---|
| (1) SB-13 | 1.2 ± 0.2⁺ | 2.36 | (2) IMPY | 5.0 ± 0.4 | 2.19 |
| 1a (SB n = 2)* | 2.9 ± 0.2 | 2.53 | 12a (IMPY n = 1) | 16 ± 2.0 | — |
| 1b (SB n = 3)⁺ | 6.7 ± 0.3 | 2.41 | 12b (IMPY n = 2) | 31 ± 9.0 | — |
| 1c (SB n = 4)⁺ | 4.4 ± 0.8 | 2.05 | 12c (IMPY n = 3) | 30 ± 2.5 | 2.69 |
| 1d (SB n = 5)⁺ | 6.8 ± 0.8 | 2.27 | 12d (IMPY n = 6) | 96 ± 14 | — |
| | | | 12e (IMPY n = 8) | 387 ± 12 | — |
| (2) PIB | 2.8 ± 0.5^{Δ} | 1.3 | (3) BF-168 | 6.4 ± 1.0^{§} | — |
| 5a (PIB n = 2) | 2.2 ± 0.5 | 3.04 | 8a (BF n = 1) | 12 ± 0.5 | — |
| 5b (PIB n = 3) | 3.8 ± 0.5 | 3.04 | 8b (BF n = 3) | 14.5 ± 5.0 | 2.93 |
| 5c (PIB n = 6) | 4.7 ± 0.9 | 2.99 | 8c (BF n = 6) | 10.0 ± 0.2 | — |
| 5d (PIB n = 8) | 9.0 ± 1.8 | — | 8d (BF n = 8) | 6.0 ± 0.6 | — |

*Values (Ki, nM) are the mean ± SEM of three independent experiments, each in duplicate.
⁺(20);
^{Δ}(6)
^{§}(15).
⁺logP = log of partition coefficient between 1-Octanol and buffer.

### Example 6

### Film Autoradiography

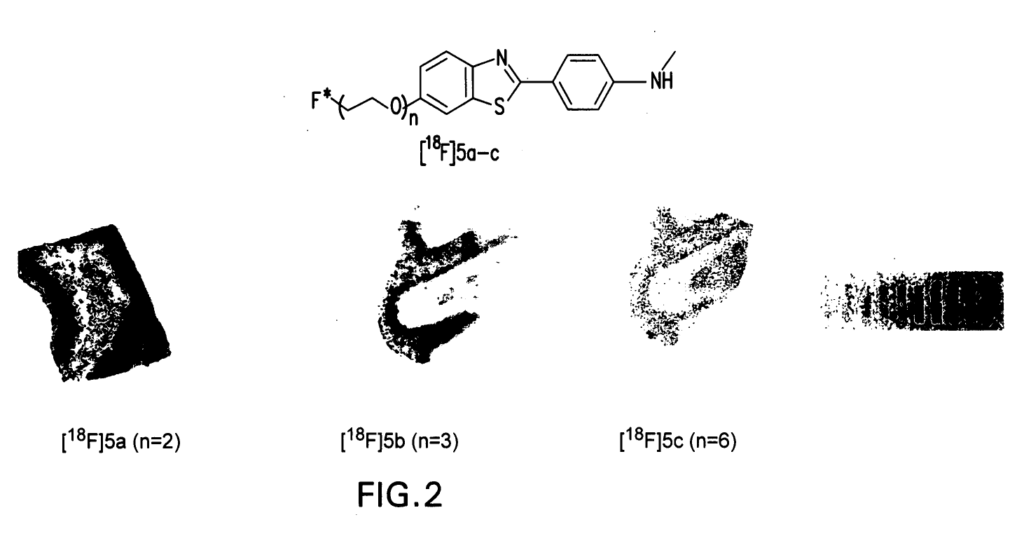

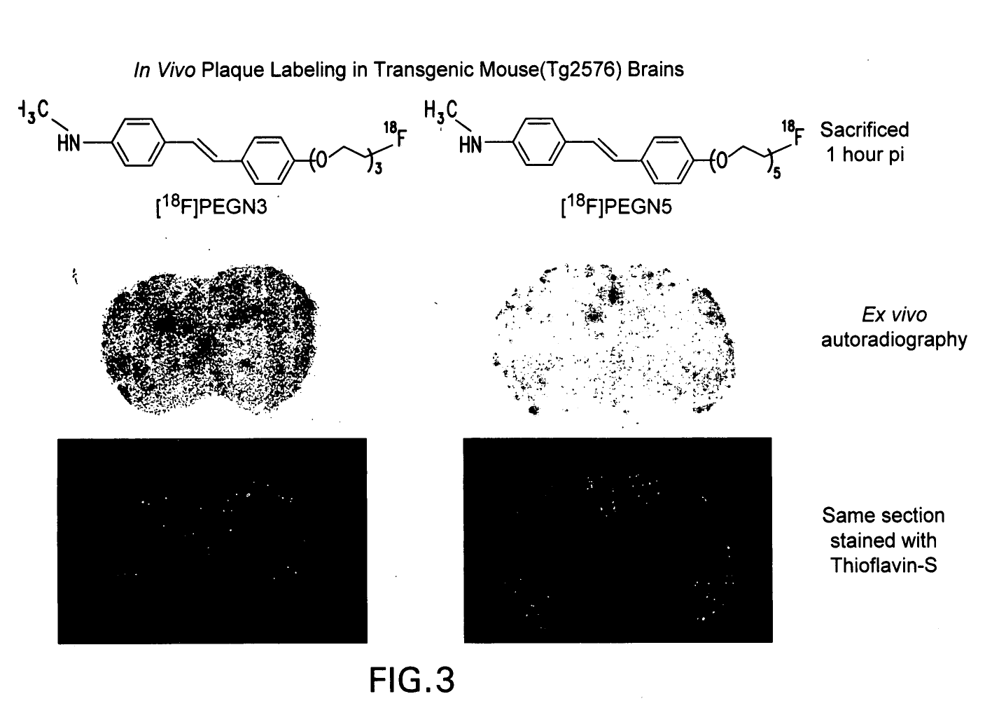

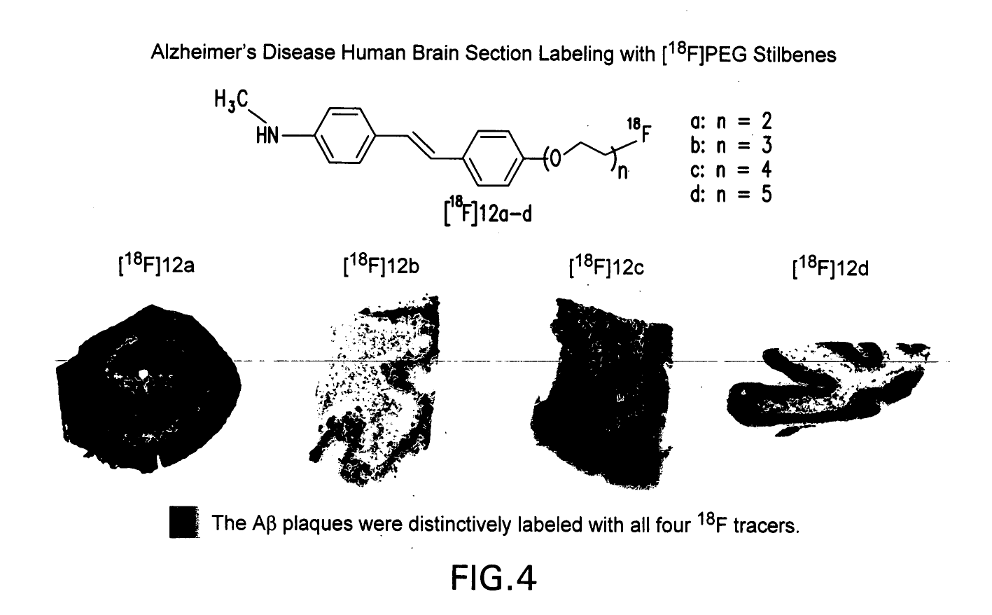

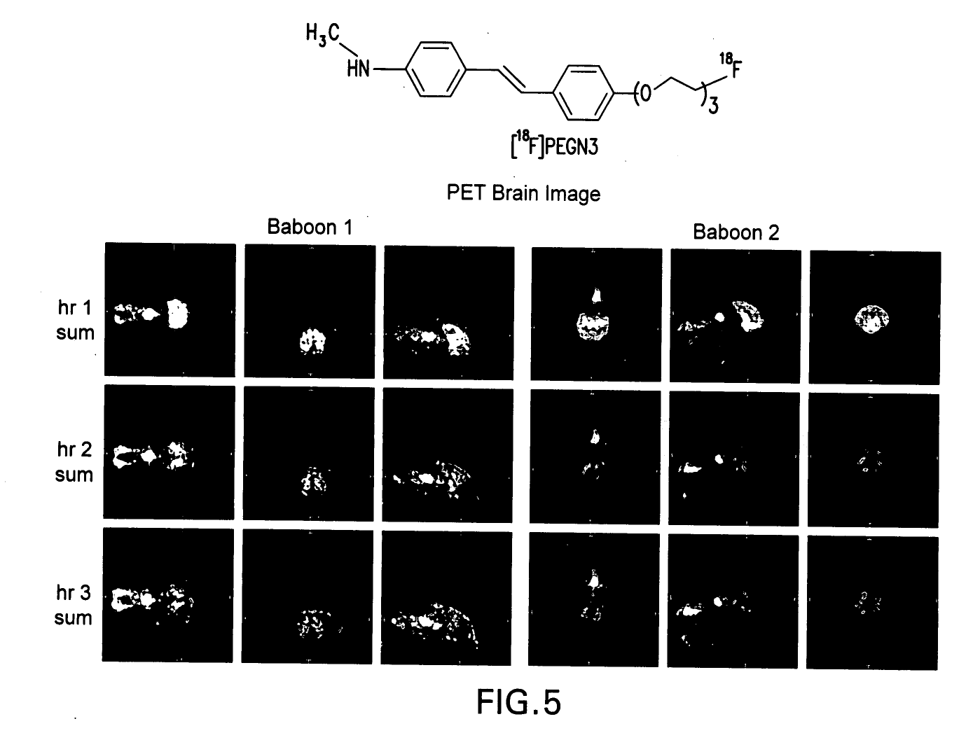

Brain sections from AD subjects were mounted onto glass slides and incubated with F-18 tracers (300,000-600,000 cpm/200 μL) for 1 hour at room temperature. The sections were then washed in saturated Li₂CO₃ in 40% EtOH (two two-min washes) and in 40% EtOH (two min) followed by rinsing with water for 30 sec. After drying, the F-18 labeled sections were exposed to Kodak MR film overnight. The results are shown in FIG. 2.

### Example 7

### Partition Coefficients

Partition coefficients were measured by mixing the [¹⁸F]tracer with 3 g each of 1-octanol and buffer (0.1 M phosphate, pH 7.4) in a test tube. The test tube was vortexed for 3 min at room temperature, followed by centrifugation for 5 min. Two weighed samples (0.5 g each) from the 1-octanol and buffer layers were counted in a well counter. The partition coefficient was determined by calculating the ratio of cpm/g of 1-octanol to that of buffer. Samples from the 1-octanol layer were re-partitioned until consistent partitions of coefficient values were obtained (usually the 3^{rd} or 4^{th} partition). The measurement was done in triplicate and repeated three times.

### Example 8

### Binding Studies

As reported previously, [¹²⁵I]IMPY (13), with 2,200 Ci/mmol specific activity and greater than 95% radiochemical purity, was prepared using the standard iododestannylation reaction and purified by a simplified C-4 mini column (13). Binding assays were carried out in 12×75 mm borosilicate glass tubes. The reaction mixture contained 50 μl of brain homogenates (20-50 μg), 50 μl of [¹²⁵I]IMPY (0.04-0.06 nM diluted in PBS) and 50 μl of inhibitors (10⁻⁵-10⁻¹⁰ M diluted serially in PBS containing 0.1% bovine serum albumin, BSA) in a final volume of 1 ml. Nonspecific binding was defined in the presence of IMPY (600 nM) in the same assay tubes. The mixture was incubated at 37° C. for 2 hr and the bound and the free radioactivity were separated by vacuum filtration through Whatman GF/B filters using a Brandel M-24R cell harvester followed by 2×3 ml washes of PBS at room temperature. Filters containing the bound ¹²⁵I ligand were assayed for radioactivity content in a gamma counter (Packard 5000) with 70% counting efficiency. Under the assay conditions, the specifically bound fraction was less than 15% of the total radioactivity. The results of inhibition experiments were subjected to nonlinear regression analysis using EBDA by which Kᵢ values were calculated.

### Example 9

### Biodistribution Studies in Normal Mice

While under isoflurane anesthesia, 0.15 mL of a saline solution containing the F-18 tracers (10-20 uCi) was injected directly into the lateral tail vein of male ICR mice. The mice (n=3 for each time point) were sacrificed by cervical dislocation at 2, 30, 60 and 120 minutes post-injection. The organs of interest were removed, weighed and assayed for radioactivity content with an automatic gamma counter. The percentage dose per organ was calculated by a comparison of the tissue counts to suitably diluted aliquots of the injected material. Total activities of blood and bone were calculated under the assumption that they were 7% and 14% of the body weight, respectively. The % dose/g of samples was calculated by comparing the sample counts with the count of the diluted initial dose. The results are shown in Table 2A-C.

TABLE 2A-C
Biodistribution in normal mice of compounds[¹⁸F]5a-c

| | | | | |
|---|---|---|---|---|
| Organ | 2 min | 30 min | 1 hr | 2 hr |

A. [¹⁸F]5a biodistribution (% dose/g, avg of 3 mice ± SD)

| | | | | |
|---|---|---|---|---|
| Blood | 3.37 ± 0.46 | 3.60 ± 0.13 | 4.55 ± 0.40 | 4.38 ± 0.14 |
| Heart | 8.32 ± 0.37 | 3.49 ± 0.27 | 3.81 ± 0.25 | 3.59 ± 0.03 |
| Muscle | 0.82 ± 0.12 | 2.83 ± 0.22 | 2.76 ± 0.17 | 2.36 ± 0.11 |
| Lung | 7.79 ± 0.34 | 3.91 ± 0.28 | 3.84 ± 0.55 | 3.63 ± 0.15 |
| Kidney | 13.02 ± 1.04 | 4.54 ± 0.62 | 3.98 ± 0.24 | 3.18 ± 0.16 |
| Spleen | 6.92 ± 0.79 | 3.93 ± 0.29 | 3.87 ± 0.61 | 3.32 ± 0.16 |
| Liver | 19.02 ± 1.06 | 7.98 ± 0.60 | 6.35 ± 0.46 | 5.05 ± 0.43 |
| Skin | 1.08 ± 0.22 | 3.14 ± 0.22 | 3.15 ± 0.28 | 2.62 ± 0.16 |
| Brain | 10.27 ± 1.30 | 4.59 ± 0.47 | 3.94 ± 0.04 | 3.86 ± 0.35 |
| Bone | 1.69 ± 0.21 | 2.28 ± 0.20 | 3.17 ± 0.39 | 6.35 ± 1.32 |

B. [¹⁸F]5b biodistribution (% dose/g, avg of 3 mice ± SD)

| | | | | |
|---|---|---|---|---|
| Blood | 6.29 ± 1.19 | 3.41 ± 0.07 | 3.91 ± 0.23 | 4.04 ± 0.45 |
| Heart | 6.26 ± 1.12 | 3.22 ± 0.33 | 3.06 ± 0.15 | 2.50 ± 0.09 |
| Muscle | 1.40 ± 0.11 | 1.92 ± 0.34 | 1.58 ± 0.13 | 1.38 ± 0.11 |
| Lung | 7.35 ± 1.50 | 3.94 ± 0.29 | 3.63 ± 0.25 | 3.24 ± 0.10 |
| Kidney | 9.02 ± 0.77 | 5.27 ± 0.77 | 3.97 ± 0.25 | 2.97 ± 0.07 |
| Spleen | 5.24 ± 0.67 | 2.66 ± 0.14 | 2.84 ± 0.13 | 2.46 ± 0.05 |
| Liver | 21.84 ± 1.56 | 13.75 ± 1.88 | 11.22 ± 0.82 | 9.13 ± 1.11 |
| Skin | 2.09 ± 0.22 | 2.27 ± 0.17 | 1.92 ± 0.13 | 1.57 ± 0.06 |
| Brain | 5.53 ± 0.56 | 2.33 ± 0.15 | 2.18 ± 0.09 | 1.96 ± 0.13 |
| Bone | 2.13 ± 0.16 | 1.48 ± 0.03 | 1.82 ± 0.04 | 2.41 ± 0.28 |

C. [¹⁸F]5c biodistribution (% dose/g, avg of 3 mice ± SD)

| | | | | |
|---|---|---|---|---|
| Blood | 3.54 ± 0.12 | 2.52 ± 0.29 | 2.46 ± 0.29 | 1.54 ± 0.19 |
| Heart | 9.37 ± 0.18 | 2.11 ± 0.27 | 1.82 ± 0.28 | 1.19 ± 0.15 |
| Muscle | 1.60 ± 0.98 | 1.87 ± 0.38 | 1.52 ± 0.24 | 0.93 ± 0.07 |
| Lung | 4.68 ± 0.27 | 2.41 ± 0.37 | 2.08 ± 0.32 | 1.24 ± 0.16 |
| Kidney | 23.00 ± 0.89 | 5.01 ± 0.84 | 3.50 ± 0.86 | 1.27 ± 0.08 |
| Spleen | 4.74 ± 0.23 | 2.05 ± 0.18 | 1.87 ± 0.26 | 1.13 ± 0.16 |
| Liver | 12.43 ± 1.11 | 3.94 ± 0.29 | 2.86 ± 0.38 | 1.67 ± 0.32 |
| Skin | 0.95 ± 0.12 | 2.14 ± 1.03 | 1.42 ± 0.14 | 0.91 ± 0.10 |
| Brain | 2.57 ± 0.12 | 1.69 ± 0.23 | 1.80 ± 0.25 | 1.29 ± 0.17 |
| Bone | 1.68 ± 0.60 | 1.20 ± 0.29 | 1.68 ± 0.17 | 2.31 ± 0.37 |

Having now fully described this invention, it will be understood to those of ordinary skill in the art that the same can be performed within a wide and equivalent range of conditions, formulations, and other parameters without affecting the scope of the invention or any embodiment thereof. All patents, patent applications, and publications cited herein are fully incorporated by reference herein in their entirety.

### BIBLIOGRAPHY

(1) Selkoe, D. J. (2000) Imaging Alzheimer's amyloid. Nature Biotechnology 18, 823-824.
(2) Hardy, J., and Selkoe, D. J. (2002) The amyloid hypothesis of Alzheimer's disease: progress and problems on the road to therapeutics. Science 297, 353-356.
(3) Golde, T. E. (2005) The Abeta hypothesis: leading us to rationally-designed therapeutic strategies for the treatment or prevention of Alzheimer disease. Brain Pathol 15, 84-7.
(4) Petkova, A. T., Leapman, R. D., Guo, Z., Yau, W.-M., Mattson, M. P., and Tycko, R. (2005) Self-Propagating, Molecular-Level Polymorphism in Alzheimer's {beta}-Amyloid Fibrils. Science 307, 262-265.
(5) Mathis, C. A., Klunk, W. E., Price, J. C., and DeKosky, S. T. (2005) Imaging technology for neurodegenerative diseases: progress toward detection of specific pathologies. Arch Neurol 62, 196-200.
(6) Mathis, C. A., Wang, Y., and Klunk, W. E. (2004) Imaging b-amyloid plaques and neurofibrillary tangles in the aging human brain. Current Pharmaceutical Design 10, 1469-1492.
(7) Shoghi-Jadid, K., Barrio, J. R., Kepe, V., Wu, H. M., Small, G. W., Phelps, M. E., and Huang, S. C. (2005) Imaging beta-amyloid fibrils in Alzheimer's disease: a critical analysis through simulation of amyloid fibril polymerization. Nucl Med Biol 32, 337-51.
(8) Shoghi-Jadid, K., Small, G. W., Agdeppa, E. D., Kepe, V., Ercoli, L. M., Siddarth, P., Read, S., Satyamurthy, N., Petric, A., Huang, S. C., Barrio, J. R., Liu, J., Flores-Torres, S., and Cole, G. M. (2002) Localization of neurofibrillary tangles and beta-amyloid plaques in the brains of living patients with Alzheimer disease: Binding characteristics of radiofluorinated 6-dialkylamino-2-naphthylethylidene derivatives as positron emission tomography imaging probes for beta-amyloid plaques in Alzheimer disease. American Journal of Geriatric Psychiatry 10, 24-35.
(9) Verhoeff, N. P., Wilson, A. A., Takeshita, S., Trop, L., Hussey, D., Singh, K., Kung, H. F., Kung, M.-P., and Houle, S. (2004) In vivo imaging of Alzheimer disease beta-amyloid with [11C]SB-13 PET. American Journal of Geriatric Psychiatry 12, 584-595.
(10) Klunk, W. E., Engler, H., Nordberg, A., Wang, Y., Blomqvist, G., Holt, D. P., Bergstrom, M., Savitcheva, I., Huang, G.-f., Estrada, S., Ausen, B., Debnath, M. L., Barletta, J., Price, J. C., Sandell, J., Lopresti, B. J., Wall, A., Koivisto, P., Antoni, G., Mathis, C. A., and Langstrom, B. (2004) Imaging Brain Amyloid in Alzheimer's Disease with Pittsburgh Compound-B. Annals of Neurology 55, 306-319.
(11) Kung, H. F., Lee, C.-W., Zhuang, Z. P., Kung, M. P., Hou, C., and Plossl, K. (2001) Novel stilbenes as probes for amyloid plaques. Journal of the American Chemical Society 123, 12740-12741.
(12) Mathis, C. A., Holt, D. P., Wang, Y., Huang, G. F., Debnath, M. L., and Klunk, W. E. (2002) 18F-labeled thioflavin-T analogs for amyloid assessment. Journal of Nuclear Medicine 43, 166P.
(13) Kung, M.-P., Hou, C., Zhuang, Z.-P., Cross, A. J., Maier, D. L., and Kung, H. F. (2004) Characterization of IMPY as a potential imaging agent for b-amyloid plaques in double transgenic PSAPP mice. European Journal of Nuclear Medicine and Molecular Imaging 31, 1136-1145.
(14) Zhuang, Z.-P., Kung, M.-P., Hou, C., Skovronsky, D., Gur, T. L., Trojanowski, J. Q., Lee, V. M.-Y., and Kung, H. F. (2001) Radioiodinated Styrylbenzenes and Thioflavins as Probes for Amyloid Aggregates. Journal of Medicinal Chemistry 44, 1905-1914.
(15) Okamura, N., Suemoto, T., Shimadzu, H., Suzuki, M., Shiomitsu, T., Akatsu, H., Yamamoto, T., Staufenbiel, M., Yanai, K., Arai, H., Sasaki, H., Kudo, Y., and Sawada, T. (2004) Styrylbenzoxazole derivatives for in vivo imaging of amyloid plaques in the brain. Journal of Neuroscience 24, 2535-2541.
(16) Zhuang, Z.-P., Kung, M.-P., Hou, C., Plossel, K., Skovronsky, D., Gur, T. L., Trojanowski, J. Q., Lee, V. M.-Y., and Kung, H. F. (2001) IBOX(2-(4′-dimethylaminophenyl)-6-iodobenzoxazole): a ligand for imaging amyloid plaques in the brain. Nuclear Medicine and Biology 28, 887-894.
(17) Kilboum, M. R. (1990) Fluorine-18 Labeling of Radiopharmaceuticals, National Academy Press, Washington, D.C.
(18) Elsinga, P. H. (2002) Radiopharmaceutical chemistry for positron emission tomography. Methods 27, 208-17.
(19) Lasne, M.-C., Perrio, C. c., Rouden, J., Barr√©, L., Roeda, D., Dolle, F. d. r., and Crouzel, C. (2002) Chemistry of β+-Emitting Compounds Based on Fluorine-18. Topics in Current Chemistry 222, 201-258.
(20) Zhang, W., Oya, S., Kung, M. P., Hou, C., Maier, D. L., and Kung, H. F. (2005) F-18 PEG Stilbenes as PET Imaging Agents Targeting Aβ Aggregates in the Brain. Nuclear Medicine and Biology 32, 799-809.
(21) Roberts, M. J., Bentley, M. D., and Harris, J. M. (2002) Chemistry for peptide and protein PEGylation. Advanced drug delivery reviews 54, 459-76.
(22) Harris, J. M., and Chess, R. B. (2003) Effect of pegylation on pharmaceuticals. Nature Review. Drug Discovery 2, 214-21.
(23) Chen, X., Hou, Y., Tohme, M., Park, R., Khankaldyyan, V., Gonzales-Gomez, I., Bading, J. R., Laug, W. E., Conti, P. S., Haubner, R., Wester, H. J., Burkhart, F., Senekowitsch-Schmidtke, R., Weber, W., Goodman, S. L., Kessler, H., and Schwaiger, M. (2004) Pegylated Arg-Gly-Asp peptide: 64Cu labeling and PET imaging of brain tumor alphavbeta3-integrin expression. Journal of Nuclear Medicine 45, 1776-83.
(24) Chen, X., Park, R., Hou, Y., Khankaldyyan, V., Gonzales-Gomez, I., Tohme, M., Bading, J. R., Laug, W. E., Conti, P. S., Haubner, R., Wester, H. J., Burkhart, F., Senekowitsch-Schmidtke, R., Weber, W., Goodman, S. L., Kessler, H., and Schwaiger, M. (2004) MicroPET imaging of brain tumor angiogenesis with 18F-labeled PEGylated RGD peptide. European Journal of Nuclear Medicine and Molecular Imaging 31, 1081-9.
(25) Zhang, W., Oya, S., Kung, M. P., Hou, C., Maier, D. L., and Kung, H. F. (2005) F-18 Stilbenes as PET Imaging Agents for Detecting beta-Amyloid Plaques in the Brain. Journal of Medicinal Chemistry 48, 5980-8.
(26) Mathis, C. A., Wang, Y., Holt, D. P., Huang, G.-f., Debnath, M. L., and Klunk, W. E. (2003) Synthesis and Evaluation of ¹¹C-Labeled 6-Substituted 2-Arylbenzothiazoles as Amyloid Imaging Agents. Journal of Medicinal Chemistry 46, 2740-2754.
(27) Barluenga, J., Bayon, A. M., and Asensio, G. (1984) A New Specific Method for the Monomethylation of Primary Amines. Journal of Chemical Society, Chemical Communications, 1334-1335.
(28) Schreiner, E. P., Wolff, B., Winiski, A. P., and Billich, A. (2003) 6-(2-adamantan-2-ylidene-hydroxybenzoxazole)-O-sulfamate: a potent non-steroidal irreversible inhibitor of human steroid sulfatase. Bioorg Med Chem Lett 13, 4313-6.
(29) Zhuang, Z. P., Kung, M. P., Wilson, A., Lee, C. W., Plossl, K., Hou, C., Holtzman, D. M., and Kung, H. F. (2003) Structure-activity relationship of imidazo[1,2-a]pyridines as ligands for detecting beta-amyloid plaques in the brain. Journal of Medicinal Chemistry 46, 237-243.
(30) Shimadzu, H., Suemoto, T., Suzuki, M., Shiomitsu, T., Okamura, N., Kudo, Y., and Sawada, T. (2004) Novel probes for imaging amyloid-b: F-18 and C-11 labeling of 2-(4-aminostyryl)benzoxazole derivatives. Journal of Labelled Compounds & Radiopharmaceuticals 47, 181-190.
(31) Kung, M.-P., Hou, C., Zhuang, Z.-P., Skovronsky, D., and Kung, H. F. (2004) Binding of two potential imaging agents targeting amyloid plaques in postmortem brain tissues of patients with Alzheimer's disease. Brain Research 1025, 89-105.

What is claimed is:

1. A method of imaging amyloid deposits comprising,
a) administering to a mammal an amount of an imaging agent, said agent comprising a Ligand (L) that binds amyloid deposits covalently attached to a moiety (X′), and having the following Formula IV, 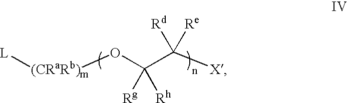
wherein,
X′ is selected from the group consisting of hydrogen, hydroxy, C₁₋₄ alkoxy, halogen, radiohalogen, 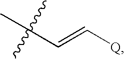
wherein Q is a halogen or radiohalogen, and a chelating moiety bound to a radio-metal;
R^{a}, R^{b}, R^{d}, R^{c}, R^{g} and R^{h} are, in each instance, independently selected from the group consisting of hydrogen, hydroxy, C₁₋₄ alkoxy, C₁₋₄ alkyl, and hydroxy(C₁₋₄)alkyl;
m is an integer from 0 to 5; and
n is an integer from 1 to 10;
b) allowing sufficient time for said agent to become associated with one or more amyloid deposits in said mammal; and
c) detecting said agent associated with said one or more amyloid deposits;
provided,
that one of X′ or Q either contains a radiohalogen or radiometal as permitted, or (L) is covalently bonded to a radiohalogen;
that in Formula IV, when m is zero, L is other than: 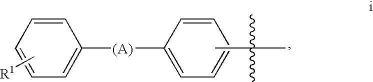
or a pharmaceutically acceptable salt thereof, wherein:
A is selected from the group consisting of: 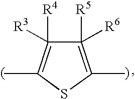
wherein R³, R⁴, R⁵, R⁶, R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl;
and 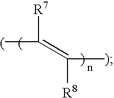
wherein n is an integer between 1 and 6; and R⁷ and R⁸ are in each instance independently selected from the group consisting of hydrogen, hydroxy, amino, methylamino, dimethylamino, C₁₋₄alkoxy, C₁₋₄ alkyl, and hydroxy(C₁₋₄)alkyl;
R¹ is selected from the group consisting of:
a. NR^{a′}R^{b′}, wherein R^{a′} and R^{b′} are independently hydrogen, C₁₋₄ alkyl or (CH₂)_{d}X, where X is halogen, and d is an integer between 1 and 4,
b. hydroxy,
c. C₁₋₄ alkoxy, and
d. hydroxy(C₁₋₄)alkyl.

2. The method of claim 1, wherein said ligand (L) has the following structure: 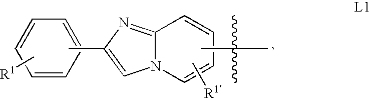
wherein,
R¹ and R^{1′}, are in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl.

3. The method of claim 1, wherein said ligand (L) has the following structure: 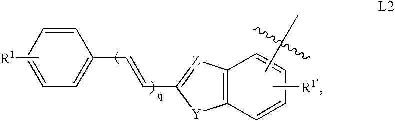
wherein
R¹ and R^{1′}, are in each instance, independently selected from the group consisting of: hydrogen, halogen, radiohalogen, C₁₋₄ alkyl hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl;
q is an integer from 0 to 3;
Z is O, S or N; and
Y is N or —CH.

4. The method of claim 3, wherein q is 0 or 1.

5. The method of claim 1, wherein said ligand (L) has the following structure: 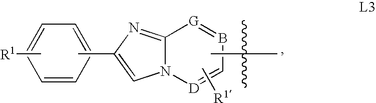
wherein,
G, B and D are CH or N,
provided that at least one no more than two of G, B and D is N; and
R¹ and R^{1′}, are in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl.

6. The method of claim 1, wherein said ligand (L) has the following structure: 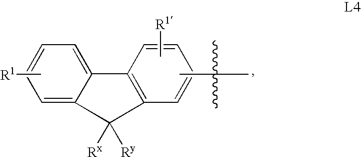
wherein,
R¹ and R^{1′} are, in each instance, independently selected from the group consisting of: hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d}R^{e}, wherein R^{d} and R^{e}, in each instance, is independently selected from the group consisting of: hydrogen, C₁₋₄ alkyl and halo(C₁₋₄)alkyl, or R^{d} and R^{e} are taken together with the nitrogen to which they are attached to form a 5- to 7-member heterocyclic ring optionally having O, S or NR⁶ in said ring, where R⁶ is hydrogen or C₁₋₄ alkyl;
R^{x} and R^{y}, in each instance, is independently selected from the group consisting of hydrogen, C₁₋₄ alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl.

7. The method of claim 1, wherein said ligand (L) has the following structure: 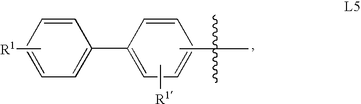
wherein,
R¹ and R^{1′} are, in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl.

8. The method of claim 1, wherein said ligand (L) has the following structure: 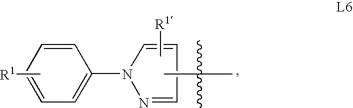
wherein,
R¹ and R^{1′} are, in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl.

9. The method of claim 1, wherein said ligand (L) has the following structure: 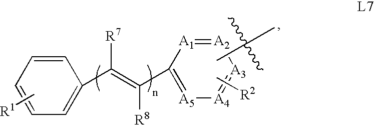
wherein,
n is an integer from one to six;
at least one, no more than three, of A₁, A₂, A₃, A₄ and A₅ is N, the others are —CH or —CR² as permitted;
R¹ and R², in each instance, are independently selected from the group consisting of hydrogen, C₁₋₄ alkyl, hydroxy, C₁₋₄ alkoxy, hydroxy(C₁₋₁₀)alkyl, amino(C₂₋₄)alkyl, halo(C₁₋₄al)yl, C₆₋₁₀ aryl, haloarylalkyl, and NR^{a′}R^{b′}(CH₂)ₚ—,
wherein p is an integer from 0 to 5, and R^{a′} and R^{b′}, in each instance, is independently selected from the group consisting of: hydrogen, C₁₋₄ allyl and halo(C₁₋₄)alkyl, or R^{a′} and R^{b′} are taken together with the nitrogen to which they are attached to form a 5- to 7-member heterocyclic ring optionally having O, S or NR⁶ in said ring, where R⁶ is hydrogen or C₁₋₄ alkyl are independently hydrogen, C₁₋₄ alkyl or (CH₂)_{d}X, where X is halogen, and d is an integer from 1 to 4, and
R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl.

10. The method of claim 1, wherein said radiohalogen is selected from the group consisting of ¹⁸F, ¹³¹I, ¹²⁵I, ¹²³I, ¹²⁴I, ⁷⁷Br and ⁷⁶Br.

11. The method of claim 10, wherein said radiohalogen is ¹⁸F.
INSERT: 11a. The method of claim 1, wherein X′ is a chelate bound to a radio-metal of Technetium, Copper, Indium, or Gallium.

12. The method of claim 1, further comprising:
d) measuring the distribution of said agent within said mammal by positron emission tomography.

13. The method of claim 1, further comprising:
d) measuring the distribution of said agent within said mammal by single photon emission tomography.

14. The method of claim 1, wherein X′ is a N₂S₂ type chelating moiety bound to a radiometal.

15. The method of claims 1, 13 or 14, wherein said radiometal is 99m-Tc.

16. The method of claim 1, wherein said amyloid deposit is located in the central nervous system of said mammal.

17. The method of claim 1, wherein said amyloid deposit is located in the brain of said mammal.

18. A method of preparing a radiolabeled ligand comprising,
a) contacting a ligand (L-(CR^{a}R^{b})ₘ), wherein R^{a}, R^{b} and m are as described above, said ligand containing a first reactive group, with a compound having the following Formula I, 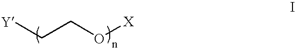
wherein n is an integer from 1 to 10, optionally from 2 to 10; Y′ is a third reactive group, and X is a second reactive group such that said first reactive group reacts with said second reactive group or the carbon to which it is attached to form a compound of Formula II, 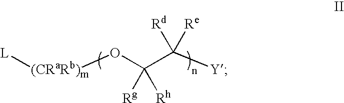
b) contacting a compound of Formula II with a reagent (Z) to prepare a compound of Formula III, 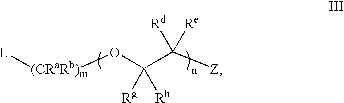
wherein Z is a leaving group;
and
c) contacting a compound of Formula III with a radiohalogenating agent, wherein a radiolabeled ligand of Formula IV as described above is prepared.

19. In a method of imaging amyloid deposits comprising:
a) administering to a mammal a first ligand capable of binding amyloid deposits in the brain;
b) allowing sufficient time for said first ligand to become associated with one or more amyloid deposits in said mammal; and
c) detecting said first ligand associated with said amyloid deposits;
the improvement comprising covalently attaching to said first ligand a group to provide a second ligand having attached thereto a radiolabel suitable for imaging without a substantial increase in the lipophilicity of said, first ligand said group having the following structure: 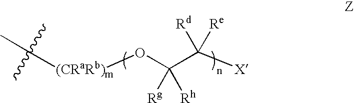
wherein R^{a}, R^{b}, R^{d}, R^{d}, R^{g}, R^{h}, m, n are as described above, and X′ is selected from the group consisting of a radiohalogen, 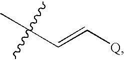
wherein Q is a radiohalogen, and a chelating moiety bound to a radio-metal; provided,
that if m is zero, said first ligand is other than: 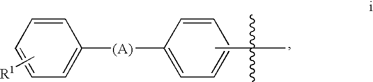
or a pharmaceutically acceptable salt thereof, wherein:
A is selected from the group consisting of: 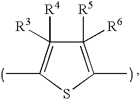
wherein R³, R⁴, R⁵, R⁶, R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl;
and 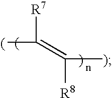
wherein n is an integer between 1 and 6; and R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl;
R¹ is selected from the group consisting of:
a NR^{a′}R^{b′}, wherein R^{a′} and R^{b′} are independently hydrogen, C₁₋₄ alkyl or (CH₂)_{d}X, where X is halogen, and d is an integer between 1 and 4,
b. hydroxy,
c. C₁₋₄alkoxy, and
d. hydroxy(C₁₋₄)alkyl.

20. A pharmaceutical composition comprising, (a) a compound capable of binding amyloid deposits, having a relatively low rate of transfer across a blood-brain barrier and having a core structure L1, L1′, L2, L2′, L3, L3′, L4, L5 L6, L6′, L7, L7′, L₈ or L9 as described herein, the improvement comprising covalently attaching a group (Z) to said compound to provide imaging compounds having increased rates of transfer across a blood-brain barrier, wherein (Z) has the following formula: 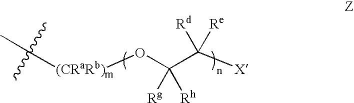
wherein R^{a}, R^{b}, R^{d}, R^{c}, R^{g}, R^{h}, m, n and X′ are as described above; and
(b) pharmaceutically acceptable diluents or excipients.

21. The method of claim 1, wherein X′ is F or ¹⁸F.

22. The method of claim 1, wherein X′ is a chelating moiety bound to a radio-metal selected from the group consisting of Technetium, Copper, Indium, Gallium or Rhenium.

23. The method of claim 1, wherein said ligand (L) has the following structure: 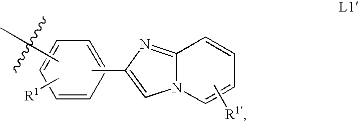
wherein,
R¹ and R^{1′}, are in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl.

24. The method of claim 1, wherein said ligand (L) has the following structure: 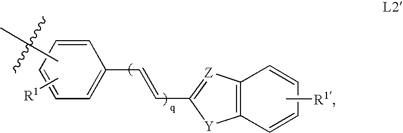
wherein
R¹ and R^{1′}, are in each instance, independently selected from the group consisting of: hydrogen, halogen, radiohalogen, C₁₋₄ alkyl hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl;
q is an integer from 0 to 3;
Z is O, S or N; and
Y is N or —CH.

25. The method of claim 1, wherein said ligand (L) has the following structure: 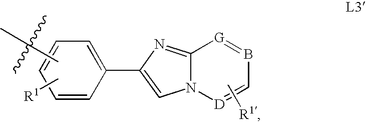
wherein,
G, B and D are CH or N,
provided that at least one no more than two of G, B and D is N; and
R¹ and R^{1′}, are in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of: hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl.

26. The method of claim 1, wherein said ligand (L) has the following structure: 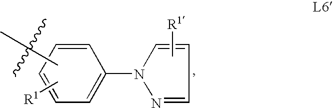
wherein,
R¹ and R^{1′} are, in each instance, independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and —NR^{d′}R^{e′}, wherein R^{d′} and R^{e′}, in each instance, is independently selected from the group consisting of hydrogen, C₁₋₄ alkyl and halo(C₁₋₄)alkyl, or R^{d′} and R^{e′} are taken together with the nitrogen to which they are attached to form a 5- to 7-member heterocyclic ring optionally having O, S or NR⁶ in said ring, where R⁶ is hydrogen or C₁₋₄ alkyl.

27. The method of claim 1, wherein said ligand (L) has the following structure: 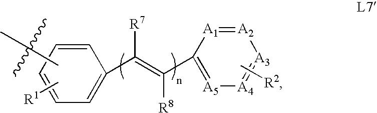
n is an integer from one to six;
at least one, no more than three, of A₁, A₂, A₃, A₄ and A₅ is N, the others are —CH or —CR²as permitted;
R¹ and R², in each instance, are independently selected from the group consisting of hydrogen, halogen, radiohalogen, C₁₋₄ alkyl hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and NR^{a′}R^{b′}(CH₂)ₚ—,
wherein p is an integer from 0 to 5, and R^{a′} and R^{b′}, in each instance, is independently selected from the group consisting of: hydrogen, C₁₋₄ alkyl and halo(C₁₋₄)alkyl, or R^{a′} and R^{b′} are taken together with the nitrogen to which they are attached to form a 5- to 7-member heterocyclic ring optionally having O, S or NR⁶ in said ring, where R⁶ is hydrogen or C₁₋₄ alkyl are independently hydrogen, C₁₋₄ alkyl or (CH₂)_{d}X, where X is halogen, and d is an integer from 1 to 4, and
R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl.

28. The method of claim 1, wherein said ligand (L) has the following structure: 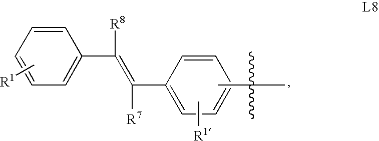
wherein,
n is an integer from one to six;
R¹ and R^{1′}, in each instance, are independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)alkyl, halo(C₁₋₄)alkyl, C₆₋₁₀ aryl, haloarylalkyl, and NR^{a′}R^{b′}(CH₂)ₚ—,
wherein p is an integer from 0 to 5, and R^{a′} and R^{b′}, in each instance, is independently selected from the group consisting of: hydrogen, C₁₋₄ alkyl and halo(C₁₋₄)alkyl, or R^{a′} and R^{b′} are taken together with the nitrogen to which they are attached to form a 5- to 7-member heterocyclic ring optionally having O, S or NR⁶ in said ring, where R⁶ is hydrogen or C₁₋₄ alkyl are independently hydrogen, C₁₋₄ alkyl or (CH₂)_{d}X, where X is halogen, and d is an integer from 1 to 4, and
R⁷ and R⁸ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl.

29. The method of claim 1, wherein said ligand (L) has the following structure: 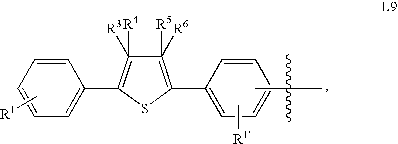
wherein,
n is an integer from one to six;
R¹ and R^{1′}, in each instance, are independently selected from the group consisting of hydrogen, halogen, radiohalogen, C₁₋₄ alkyl, hydroxy, C₁₋₄ alkoxy, hydroxy(C₁₋₁₀)alkyl, amino(C₂₋₄)allyl, halo(C₁₋₄)alkyl, C₆₋₁₀ aryl, haloarylalkyl, and NR^{a′}R^{b′}(CH₂)ₚ—,
wherein p is an integer from 0 to 5, and R^{a′} and R^{b′}, in each instance, is independently selected from the group consisting of: hydrogen, C₁₋₄ alkyl and halo(C₁₋₄)alkyl, or R^{a′} and R^{b′} are taken together with the nitrogen to which they are attached to form a 5- to 7-member heterocyclic ring optionally having O, S or NR⁶ in said ring, where R⁶ is hydrogen or C₁₋₄ alkyl are independently hydrogen, C₁₋₄ alkyl or (CH₂)_{d}X where X is halogen, and d is an integer from 1 to 4, and
R³, R⁴, R⁵ and R⁶ are in each instance independently selected from the group consisting of hydrogen, hydroxy, amino, methylamino, dimethylamino, C₁₋₄ alkoxy, C₁₋₄ alkyl, and hydroxy(C₁₋₄)alkyl.
